(19) United States
(12) Patent Application Publication
(10) Pub. No.: US 2024/0424163 A1
(43) Pub. Date: Dec. 26, 2024

(54) FUNCTIONALIZED, DECELLULARIZED CORNEAL EXTRACELLULAR MATRIX HYDROGELS FOR OCULAR TISSUE TREATMENT

(71) Applicant: THE BOARD OF TRUSTEES OF THE UNIVERSITY OF ILLINOIS, Chicago, IL (US)

(72) Inventors: Ali R. DJALILIAN, Chicago, IL (US); Ghasem YAZDANPANAH, Chicago, IL (US)

(21) Appl. No.: 18/686,954

(22) PCT Filed: Sep. 2, 2022
(86) PCT No.: PCT/US2022/042420

§ 371 Date: Feb. 27, 2024

Related U.S. Application Data
(60) Provisional application No. 63/240,054, filed on Sep. 2, 2021.

(51) Int. Cl. A61L 24/00

(52) U.S. Cl. CPC A61L 24/0031; A61L 2430/16

(57) ABSTRACT

Disclosed is a thermoresponsive, in situ curable hydrogel composition composed of decellularized corneal extracellular matrix functionalized with an acrylate in admixture with a photo-initiator, said hydrogel composition being of use in the treatment of an ocular surface wound.

### INTRODUCTION

This application claims benefit of priority to U.S. Provisional Patent Application Ser. No. 63/240,054, filed Sep. 2, 2021, the content of which is incorporated herein by reference in its entirety.

This invention was made with government support under grant nos. EY024349 and EY01792 awarded by the National Institutes of Health. The government has certain rights in this invention.

### BACKGROUND

There is a growing demand in the clinical practice for in situ rapid repair of tissues. Several hydrogels are currently available including, e.g., PHOTOCOL® (methacrylated Type I collagen), PHOTOHA® (methacrylated hyaluronic acid), LiQD Cornea (short collagen-like peptides conjugated with polyethylene glycol and mixed with fibrinogen) and GelCORE or GelMA (gelatin methacrylate). However, these conventional hydrogels are not representative of corneal macromolecular structure. Further, while fibrin glue has been used off-label in the treatment of corneal perforations, this bio-adhesive is not user-friendly in ocular and peri-ocular applications.

Therefore, ophthalmologists generally prefer suturing for ocular tissue closure, which is time-consuming, uncomfortable for patients, and can induce corneal astigmatism. Therefore, there is a need in the art for improved biomaterials that are representative of ocular tissue, ready-to-apply, easy to use, and facilitate treatment, tissue engineering, or wound healing of ocular tissue. The present invention meets this need in the art.

### SUMMARY OF THE INVENTION

This invention is a thermoresponsive, in situ curable hydrogel composition composed of decellularized corneal extracellular matrix functionalized with an acrylate (e.g., at a weight ratio in the range of 1:2 to 2:1) in admixture with a photo-initiator. In some aspects, the acrylate is a methyl acrylate, ethyl acrylate, 2-chloroethyl vinyl ether, 2-ethylhexyl acrylate, hydroxyethyl methacrylate, butyl acrylate, butyl methacrylate or trimethylolpropane triacrylate. In other aspects, the composition further includes cells, exosomes or a therapeutic agent. The invention also provides a kit and method for preparing the thermoresponsive, in situ curable hydrogel composition by functionalizing decellularized corneal extracellular matrix with an acrylate; and combining the same with a photo-initiator. A method for treating an ocular surface wound by administering and curing the thermoresponsive, in situ curable hydrogel composition is also provided as is an ocular device including the cured hydrogel composition. In some aspects, the device has an adhesion strength of at least 10 kPa, is in the form of a lens or bandage, or is 3D printed.

### BRIEF DESCRIPTION OF THE DRAWINGS

FIG. 1 shows a strain-stress plot of compression tests for light-curable cornea matrix (LC-COMatrix) hydrogels with different degrees of functionalization.

FIG. 2 shows viscosity of LC-COMatrix hydrogels of different degrees of functionalization as compared to a viscoelastic material and fibrin glue. Viscosity measurements were taken under different shear rates and temperatures (12° C., 25° C. and 37° C.).

FIG. 3 shows the fluorescent intensity of anti-Ki-67, which is representative of Ki-67 proliferation marker expression in different regions of cornea relative to the wounded area. *P<0.05, ***P<0.001.

### DETAILED DESCRIPTION OF THE INVENTION

This invention provides a thermoresponsive, in situ curable hydrogel composition and kit containing the same, wherein said hydrogel is prepared by functionalizing decellularized corneal extracellular matrix with an acrylate; and combining the functionalized, decellularized corneal extracellular matrix with a photo-initiator. The hydrogel composition of this invention now provides a ready-to-use biomaterial that is representative of native ocular tissue, has an appropriate consistency and cohesion before cross-linking to prevent spreading to adjacent areas following administration, and is suitable for applications in corneal or ocular repair and regeneration.

As used herein, the term “thermoresponsive” refers to capacity of a material to exhibit an altered physical dependent characteristic, which is on temperature. Particularly relevant herein are materials that are soluble or in a liquid state at a first temperature range (e.g., below body temperature, approximately 37° C.) and insoluble or in a solid state at a second temperature (e.g., at or above body temperature). In accordance with this invention, the thermoresponsive hydrogel is soluble or in a liquid state when stored at 4° C., 15° C. or at room temperature, but solidifies upon administration to patient.

A material that is “curable” or “cured,” or more particularly “photocurable” or “photocured,” refers to a material that is cross-linked by exposure to a specific wavelength of light thereby hardening or stiffening the material. In some instances, the material that is curable or to be cured is referred to as a “precursor.”

The hydrogel of this invention is composed of decellularized corneal extracellular matrix functionalized with an acrylate. As used herein, “decellularized corneal extracellular matrix” refers to biological material that no longer includes cellular components such as organelles, membranes, or cytoplasm. In so far as the decellularized extracellular matrix is derived from cornea tissue, or alternatively other ocular or peri-ocular tissue (e.g., lens, sclera, vitreous humor, eyelids, retina, optic nerve, and peri-ocular muscle and fat), the extracellular matrix of the hydrogel provides a material structurally similar to natural cornea tissue. In certain aspects, the decellularized corneal extracellular matrix includes various collagen types (not just collagen type I) as well as wound healing mediators including lumican, keratocan and laminin. In addition, even if the decellularized material is obtained from one animal and transplanted into a different animal, a heterologous immune reaction is restrained. Therefore, the kind of animals from which cornea tissue is collected is not particularly limited and can be collected from human sources (e.g., cadavers) or other animals. On the other hand, since it is preferable that the cornea tissue be readily available, the cornea tissue is preferably collected from an animal other than human, preferably domestic animals. Such domestic animals include cattle, horses, camels, llama, donkey, yak, sheep, pigs, goats, deer, alpacas, dogs, raccoon dogs, weasels, foxes, cats, rabbits, hamsters, guinea pigs, rats, mice, squirrels, raccoons, and the like. Among these, biological tissues from pigs and rabbits are preferable in view of stable availability.

The decellularized corneal extracellular matrix of the instant hydrogel is prepared by harvesting a suitable tissue, for example ocular and peri-ocular tissues, preferably cornea, from porcine and human cadavers and removing the cellular components of the tissue. Ideally, cellular components are removed by treatment with one or more proteases followed by multiple cycles (e.g., 6, 7, 8, 9, or more) of freeze (−80° C.) and thaw (37° C.). Alternatively, cellular components are removed by treatment of the tissue with ammonium hydroxide, a surfactant and chelator. Nucleic acids are subsequently removed from the tissue by treatment with one or more nucleases, and the bio-burden is decreased by, e.g., the addition of ethanol and peracetic acid. Following freeze-drying and milling or pulverizing, the resulting fine powder of decellularized extracellular matrix is partially digested, e.g., with protease (e.g., pepsin) under acidic conditions. At the end of digestion, when tissue particles are no longer present, the pH of the material is neutralized to provide a thermoresponsive hydrogel.

To functionalize the thermoresponsive hydrogel, decellularized corneal extracellular matrix is reacted with at least one acrylate moieties. This modification means that the components of the decellularized corneal extracellular matrix are able to be bound to each other by crosslinking, in particular photocrosslinking, using light via a photoinitiator to form hydrogels.

An “acrylate” is used in the broadest sense and, in most cases, refers to derivatives of acrylic acids of Formula (I): R¹CHC(R²)C(O)OR³, wherein R¹, R² and R³ are independently H, C₁-C₁₀ alkyl, C₂-C₁₀ alkenyl, C₂-C₁₀ alkynyl, cycloalkyl, heterocycloalkyl, aryl or heteroaryl, each of which is optionally substituted. In some cases, the term “acrylate” refers to derivatives of Formula (II): R¹CHC(R²)OR³. Further, the backbone of the alkyl, alkenyl or alkynyl can be interspersed with one or more of O, S, or NH. This is known as a “heteroalkyl” group. In some aspects, R¹ can be H, methyl, ethyl or propyl. In other aspects, R² in the compounds of Formula (I) can be H, methyl, ethyl or propyl. The term “methacrylate” usually refers to a specific acrylate derivative of Formula (Ia): CH₂C(CH₃)C(O)O—, i.e., an acrylate of Formula (I) when R¹ is H and R² is methyl. Examples of suitable acrylates for use in the present invention are methyl acrylate, ethyl acrylate, 2-chloroethyl vinyl ether, 2-ethylhexyl acrylate, hydroxyethyl methacrylate, butyl acrylate, butyl methacrylate and trimethylolpropane triacrylate.

The decellularized corneal extracellular matrix can be modified with one or more acrylates using any suitable available means. For example, decellularized corneal extracellular matrix can be acrylated by the addition of an acrylate anhydride to a solution including decellularized corneal extracellular matrix in buffer and allowing the reagents to react for a sufficient period of time and at an appropriate temperature. A procedure such as this is described in Example 1.

Notably, the properties of the hydrogel of the present invention can be controlled by controlling the degree of functionalization, i.e., acrylation. In order to produce a hydrogel in accordance with the present invention, a person skilled in the art will understand that the acrylate moieties need to be present on decellularized corneal extracellular matrix in proportions that are sufficient to crosslink the modified decellularized corneal extracellular matrix such that a hydrogel can be formed in the presence of water. The degree of acrylation may be defined as (the number of acrylated lysines/the total lysines in the decellularized corneal extracellular matrix)×100. Accordingly, the degree of acrylation can range from about 1% (where, for example, 1 out of every 100 lysine groups is acrylated) to about 100% (i.e., where all available lysine groups are acrylated). During the preparation of the acrylated decellularized corneal extracellular matrix, it was observed that a suitable hydrogel is formed when the ratio of decellularized corneal extracellular matrix to acrylate is, by weight, in the range of 1:2 to 2:1. Accordingly, in some aspects, the weight ratio of the decellularized corneal extracellular matrix to acrylate is in the range of 1:2 to 2:1. In certain aspects, the weight ratio of the decellularized corneal extracellular matrix to acrylate is at least 1:1 or 2:1.

In some aspects, the thermoresponsive hydrogel comprises or consists of decellularized corneal extracellular matrix functionalized with an acrylate. Accordingly, while other structural components may be added to the decellularized corneal extracellular matrix (and cross-linked therein), preferably the hydrogel itself is composed solely of decellularized corneal extracellular matrix functionalized with an acrylate. Examples of other structural components that may be added to the thermoresponsive hydrogel either before or after curing include, e.g., silk derivatives, alginate, polyvinyl alcohol (PVA), polyethylene glycol (PEG), poly(2-hydroxyethyl methacrylate) (pHEMA), poly(acrylamide), poly(methacrylamide), poly(methyl methacrylate) (PMMA), poly(lactide-co-trimethylene carbonate) (PTMC), polyfumarate, poly (lactic acid) (PLA), polycaprolactone (PCL), poly (N-vinyl-2-pyrrolidone), alginate, hyaluronan, heparin, silk sericin, methylcellulose, gellan gum, chondroitin sulfate, chitosan.

Advantageously, the instant hydrogel is both

biocompatible and biodegradable. As used herein, the term “biocompatible” refers to materials that are not toxic to cells or organisms. In some aspects, a substance is considered to be “biocompatible” if its addition to cells in vitro results in less than or equal to approximately 10% cell death, usually less than 5%, more usually less than 1%, and preferably less than 0.1%. The term “biodegradable,” as used to describe the polymers, hydrogels, compositions, and/or wound dressings that are degraded or otherwise “broken down” under exposure to physiological conditions. In some aspects, a biodegradable material is broken down by cellular machinery, enzymatic degradation, chemical processes, hydrolysis, etc.

To facilitate cross-linking of the decellularized corneal extracellular matrix functionalized with an acrylate, this invention further provides the use of a photoinitiator. A “photoinitiator,” as used herein, is a compound or combination of compounds capable of converting absorbed light energy, generally or especially UV or visible light, into chemical energy in the form of initiating species, e.g., free radicals or cations. Based on the mechanism by which initiating radicals are formed, photoinitiators are generally divided into two classes: Type I photoinitiators, which undergo a unimolecular bond cleavage upon irradiation to yield free radicals; and Type II photoinitiators, which undergo a bimolecular reaction where the excited state of the photoinitiator interacts with a second molecule (a co-initiator) to generate free radicals. UV photoinitiators of both Type I and Type II are known whereas visible light photoinitiators generally belong to the Type II class. Such “initiating species” serve to initiate polymerization in a suitable photopolymerizable material, in this case, a photopolymerizable material. The photoinitiators may be in particular aspects water soluble, inhibited by oxygen, and are preferably biocompatible.

Any suitable photoinitiator or combination of photoinitiators may be used in the invention so long as upon photoactivation cross-linking of the acrylated decellularized corneal extracellular matrix is initiated. In certain aspects, the photoinitiator includes, but are not limited to, at least one of an camphorquinone, fluorescein, riboflavin, eosin Y, acetophenone, anisoin, an anthraquinone, a sodium salt of anthraquinone-2-sulfonic acid, benzil, benzoin, a benzoin ether (e.g., ethyl, methyl, isopropyl, isobutyl ether), benzophenone, 3,3′, 4, 4′-benzophenonetetracarboxylic dianhydride, 4-benzoylbiphenyl, 2-benzyl-2-(dimethylamino)-4′-morpholinobutyrophenone, 4,4′-bis(diethylamino)benzophenone, 4,4′-bis(dimethylamino)benzophenone, camphorquinone, 2-chlorothioxanthen-9-one, dibenzosuberenone, 2,2-diethoxyacetophenone, 4,4′-dihydroxybenzophenone, 2,2-dimethoxy-2-phenylacetophenone, 2,2-dimethoxy-1, 2-diphenylethan-1-one, 4-(dimethylamino)benzophenone, 4,4′-dimethylbenzil, 2,5-dimethylbenzophenone, 3,4-dimethylbenzophenone, 4′-ethoxyacetophenone, 2-ethylanthraquinone, fluorescein, hydroxyacetophenone, 3-hydroxybenzophenone, 4-hydroxybenzophenone, 1-hydroxycyclohexyl phenyl ketone, 2-hydroxy-2-methylpropiophenone, 2-mercaptothioxanthone, 2-methylbenzophenone, 3-methylbenzophenone,methybenzoylformate, 2-methyl-4′-(methylthio)-2-morpholinopropiophenone, 4′-phenanthrenequinone, phenoxyacetophenone, or a thioxanthen-9-one. Also useful in the practice of the invention are photoinitiators having two initiators linked by a short polymer backbone, e.g., benzoin polydimethyl siloxane Benzoin (B-pdms-B) wherein two benzoin moieties are linked by a dimethyl siloxane bridge. In some cases, the photoinitiator may also be associated with a sensitizer. Suitable sensitizers include p-(dialkylamino aldehyde); n-alkylindolylidene; and bis [p-(dialkyl amino) benzylidene] ketone.

In preferred aspects, the photoinitiator compound comprises Eosin Y, Eosin B or fluorescein. Eosin Y is most commonly known as a water soluble xanthene dye. Eosin Y is a Type II photoinitiator that is typically used in combination with triethanolamine (TEOA). However, as with other Type II photoinitiators, any suitable co-initiator can be used. Having an absorption peak around 514 nm, Eosin Y is activated efficiently by low-toxicity, visible (green) light. Notably, Eosin Y itself has been shown to exhibit biocompatibility in a range of applications. In some aspects, Eosin Y is used in combination with a co-monomer. An exemplary combination is Eosin Y as a photoinitiator, Triethanolamine as a co-initiator and N-vinylcaprolactam as a co-monomer.

The composition of this invention, which is composed of a combination of the decellularized corneal extracellular matrix functionalized with an acrylate and photoinitiator, may be a solid (e.g., powder) or a liquid composition containing the components mentioned above. In some aspects, other components, such as one or more pharmaceutically acceptable excipients, one or more therapeutic agents, as well as any of the other additives (e.g., swelling agents), to assist in the repair and/or restoration of the target ocular tissue (and/or to achieving targeted delivery of therapeutic compounds), will also be included in the compositions of the present invention. A powder composition may be reconstituted or converted to a hydrogel by exposure to an aqueous environment. For example, the powdered composition may be added to a mold, followed by addition of an aqueous solution (such as a buffer or saline solution). The powdered composition may also be provided directly to the tissue repair site, where it will absorb water from the surrounding environment to form a hydrogel at the site, and/or may be provided to the site, followed by addition of an aqueous solution to the composition to form the hydrogel in situ. In certain aspects, the composition is provided in the form of a curable liquid.

As an alternative to photocrosslinking, the hydrogel may be chemically crosslinked. Examples of suitable chemical cross-linkers include, but is not limited, to EDC-NHS (1-ethyl-3-(3-(dimethylamino)propyl) carbodiimide and N-hydroxy-succinimide), polyrotaxane multiple aldehyde (PRA) crosslinkers, strain-promoted azide-alkyne cycloaddition (SPAAC), a copper-free form of click chemistry or any other commercial cross linker.

In some aspects, the hydrogels of this invention are formed in situ, enabling the hydrogel to conform to the shape of the implantation site. In accordance with this aspect, a thermoresponsive in situ curable hydrogel composition (i.e., a precursor solution including the hydrogel and photoinitiator, kept at a temperature below 37° C. prior to use) is delivered to a target site by injection. Upon exposure to body temperature, the thermoresponsive in situ curable hydrogel solidifies and light of an appropriate wavelength and duration is applied to the in situ curable hydrogel composition resulting in cross-linking of the hydrogel and in situ formation of a cured hydrogel that is tailored to the shape of the target site. This is particularly useful in wound healing applications.

For applications including in situ curing, the hydrogel may be administered in liquid form via, e.g., a double or single barrel syringe, cannula, vial, pipette, or squeeze tube. In some instances, the hydrogel can be spread using a sterile applicator to be flush with the wound or mounded within and around the wound site to create a scaffold that extends beyond the wound site or tissue defect to provide additional protection, moisture, and structure to support tissue regeneration.

In other aspects, the hydrogel is cured prior to use to provide an ocular device for ocular tissue engineering and repair. The hydrogel may be formed, cast, molded, or three-dimensionally (3D) printed in the shape of a film, sheet, tube, or other 3D shape. The hydrogel can be formed, cast, molded, or printed into the desired shape in liquid form, solidified by elevating the temperature to at or above 37° C. and cross-linked by exposure to light to retain the desired shape. When formed as a sheet, the sheet can be flat or have curvatures to closely match the contours of the injured, damaged, or diseased tissue being repaired, replaced, or regenerated. The device may be of any geometrical shape, including but not limited to squares, rectangles, trapezoids, triangles, circles, ellipses, spheres and the like. In certain aspects, the ocular device may be in the form of a lens (e.g., scleral contact lens) or bandage. The ocular device can be formed, cast, molded or printed at a thickness in the range of about 0.1 μm to about 10 mm, about 1 μm to about 1 mm, or about 1 μm to about 2 mm, or any intervening range thereof.

The light for curing can be delivered via a wide spectrum white light (incandescent or LED), a green light, blue LED light, and/or UV light depending on the photoinitiator. A flashlight, wand, lamp, or even ambient light may be used to supply the white light. Exposure should occur between 0.1 seconds and 15 minutes, preferably between 30 seconds and 10 minutes, most preferably between 1 minute and 5 minutes. The intensity of light should range between 0.01 μW/cm² and 1000 μW/cm², preferably 1 μW/cm² and 200 μW/cm², most preferably 50 μW/cm² and 100 μW/cm², at the site of annealing. For aspects in which light initiated annealing is used, the hydrogel may be stored in an opaque (opaque with respect to wavelength range that initiates annealing) container prior to use.

Advantageously, the mechanical and/or chemical properties of the hydrogel of this invention can be tailored or tuned to a particular application. Mechanical and/or chemical properties that can be tuned include tensile strength, compression strength, flexural strength, modulus, elongation, or toughness of the hydrogel. One or more of these properties can be modulated by, e.g., the degree of functionalization of the decellularized corneal extracellular matrix, the concentration of photoinitiator in the hydrogel, the intensity of the light source, and/or the duration of the curing. In some aspects, the entire hydrogel is composed of photoinitiator-compatible polymers thereby ensuring that the photoinitiator is the limiting reagent in the crosslinking process. In other aspects, use of a higher intensity light source over longer periods provides more energy to release free radicals, leading to a firmer gel. In accordance with particular aspects of this invention, cross-linking is controlled by the degree of functionalization of the decellularized corneal extracellular matrix. Ideally, the hydrogel has a degree of functionalization is in the range of about 10% to about 100%, preferably about 15% to about 90%, more preferably about 35% to 80% or most preferably about 65% to 75% or any intervening range thereof.

In some aspects, the hydrogel is prepared in manner suitable for use in ocular wound healing applications. In this respect, certain aspects provide for an in situ curable hydrogel having robust cohesion prior to cross-linking to limit spread into an undesired area during treatment. In accordance with this aspect, the in situ curable hydrogel has a viscosity of at least 100 PaS or preferably 1000 Pas at 25° C. at a shear rate of 0.01 s⁻¹.

In other aspects, the invention provides for a cured hydrogel or ocular device having an adhesion strength of at least 2 kPa, or preferably at least 4 kPa, or more preferably at least 10 kPa, or most preferably at least 15 kPa or 20 kPa.

In further aspects, the invention provides for a cured hydrogel or ocular device having viscoelastic characteristics. In accordance with this aspect, the cured hydrogel or ocular device has a G′ (which measures the elastic component) in the range of 400 Pa and 10000 Pa, preferably 2000 Pa and 9000 Pa, or more preferably 7200 Pa and 8400 Pa; and a G″ (which measures the plastic component) in the range of 30 Pa and 400 Pa, preferably 120 Pa and 300 Pa, or more preferably 200 Pa and 300 Pa each measured at frequency of 0.159 Hz and 5% strain for 1 minute.

For ocular applications, it is preferable that the cured hydrogel is sufficiently strong enough to seal and repair injuries in the range of 1 to 6 mm in size (e.g., length or diameter). Accordingly, in some aspects, the cured hydrogel exhibits a burst pressure of at least 50 mmHg, 100 mmHg, 120 mmHg, 140 mmHg, 160 mmHg, 180 mmHg, 200 mmHg, 220 mmHg, 240 mmHg, 260 mmHg, 280 mmHg, 300 mmHg, 320 mmHg, 340 mmHg, 360 mmHg, 380 mmHg, 400 mmHg, 420 mmHg, 440 mmHg, 460 mmHg, 480 mmHg, 500 mmHg, 520 mmHg, or 540 mmHg.

The hydrogels described herein are also swellable. The term refers hydrogels “swellable” to that are substantially insoluble in a swelling agent and are capable of absorbing a substantial amount of the swelling agent, thereby increasing in volume when contacted with the swelling agent. As used herein, the term “swelling agent” refers to those compounds or substances which produce at least a degree of swelling. Typically, a swelling agent is an aqueous solution or organic solvent, however the swelling agent can also be a gas. In some aspects, a swelling agent is water or a physiological solution, for example phosphate buffer saline, or growth media. In some aspects, the hydrogel composition and/or ocular device includes a swelling agent. In some aspects, the hydrogel can contain over 50% (w/v), over 60% (w/v), over 70% (w/v), over 80% v, over 90% (w/v), over 91% (w/v), over 92% (w/v), over 93% (w/v), over 94% (w/v), over 95% (w/v), over 96% (w/v), over 97% v, over 98% (w/v), over 99% (w/v), or more of the swelling agent.

Exemplary hydrogels and devices constructed in accordance with the present disclosure can be stored for at least 4 months at approximately 4° C., or up to one year or more at approximately −20° C.

This invention also provides a method for treating an ocular surface wound by administering the thermoresponsive, in situ curable hydrogel composition or ocular device of the invention to the ocular surface wound and optionally applying light to cure the hydrogel. By “treatment” or “treating” is correcting, reinforcing, reconditioning, remedying, making up for, making sound, renewing, mending, or patching an ocular surface wound to facilitate restoration of the tissue and preferably function.

The hydrogel composition or ocular device may be administered using any amount and any route of administration effective for treatment. The exact amount required will vary from subject to subject, depending on the species, age, and general condition of the subject, the severity of the wound, the particular hydrogel, its mode of administration, its mode of activity, and the like.

Exemplary wounds that can be treated by the compositions or devices described herein include blast injuries suffered during combat such as blunt trauma, shrapnel wounds and burns; burns; cuts (superficial and deep, such as those made during surgery); scrapes; abrasions; gashes; punctures and macro-perforations with tissue loss.

The hydrogel or device of this invention may also include cells to assist in repair and/or regeneration of the target ocular tissue. The hydrogels of the present invention are particularly advantageous in that they preserve cells in a viable state. The cell populations can be developmentally mature or restricted, developmentally potent or plastic, or a combination of the foregoing cell types. Examples of cells include stem cells or precursor cells.

In one aspect, a hydrogel or ocular device including a hydrogel can be molded into any suitable form or shape, as discussed above. The hydrogel or ocular device containing the hydrogel or a composition for forming a hydrogel, is formed in a subject, in a wound, or in an area of space where new ocular tissue is needed. In some aspects, one or more cell populations may be mixed with the curable hydrogel and the device formed in situ. Therefore, hydrogels or devices including a hydrogels of the present invention, and further comprising one or more populations of cells growing thereon, can accelerate tissue growth and regeneration and participate as a reinforcing material in a newly constructed, cell-based ocular tissue.

The hydrogel or device of the present invention may also include other components such as pharmaceutically acceptable excipients, exosomes, and therapeutic agents (for example drugs, vitamins and minerals), to assist in repair and/or re-generation of the target tissue, and/or to provide a method of achieving targeted delivery of therapeutic agents. In some aspects, the hydrogels or devices may include cells that natively express, or that are genetically modified to express, a particular extracellular material, cytokine, and/or growth factor to promote or facilitate the repair, restoration, regeneration, or replacement of a tissue or organ.

Any therapeutic agent known to a person skilled in the art to be of benefit in the treatment of an ocular surface wound or other ocular disease is contemplated as a therapeutic agent in the context of the present invention. Therapeutic agents include growth factors, enzymes, DNA, plasmid DNA, RNA, siRNA, proteins, lipids, antibodies, antibiotics, anti-inflammatory agents, anti-sense nucleotides and transforming nucleic acids or combinations thereof. Any of the therapeutic agents can be combined to the extent such combination is biologically compatible. Suitable growth factors include, but are not limited, endothelial growth factor (VEGF), bone morphogenic protein (BMP), epidermal growth factor (EGF), brain derived neurotrophic factor (BDNF) and transforming growth factor (TGF).

Pharmaceutically acceptable excipients include any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Except insofar as any conventional excipient is incompatible with a substance or its derivatives, such as by producing any undesirable biological effect or otherwise interacting in a deleterious manner with any other component(s) of the hydrogel, its use is contemplated to be within the scope of this invention.

To facilitate the preparation and delivery of the thermoresponsive, in situ curable hydrogel composition, this invention also provides a kit or “article of manufacture,” wherein said kit or article of manufacture includes decellularized corneal extracellular matrix functionalized with an acrylate and at least one photo-initiator. A kit may also include, for example, a slit lamp green light source or simple battery powered LED light source. In addition, a kit may further include other materials desirable from commercial and user standpoint, including other buffers, diluents, filters, needles, and syringes. Kits typically include instructions for use of the hydrogels of the present invention. Instructions may, for example, protocols and/or a description about conditions for cross-linking and forming a cured hydrogel from the composition, administration of hydrogels to a subject in need thereof, production of hydrogel assemblies, etc. Kits will generally include one or more vessels or containers so that some or all of the individual components and reagents may be separately housed.

### Example 1: Tunable, Light-Curable Cornea Matrix (LC-COMatrix) for In-Situ Repair of Corneal Macro-Perforations and Stromal Defects

Harvesting and Decellularization of Porcine Corneas. Porcine eyeballs were obtained from a certified abattoir (Park Packing Co., Chicago, IL), soaked in phosphate-buffered saline (PBS) containing 2% gentamicin, and corneas were excised. The harvested corneas were cut into pieces and washed with PBS and pure water. Porcine cornea tissue fragments were transferred to a 50 ml conical tube containing 35 ml of 10 mM Tris-HCl plus protease inhibitor cocktail the manufacturer's recommended concentration (complete™, EDTA-free Protease Inhibitor Cocktail, Roche). The tissue fragments underwent nine cycles of freeze (−80° C.) and thaw (37° C.) in 9 days (one cycle per day). After freeze-thawing, the tissue fragments were washed with pure water and incubated in DNAse (50 mM Tris-HCl containing 7.5 U/ml deoxyribonuclease (Sigma)) for 16 hours at 37° C. The bio-burden of the decellularized porcine corneas was then decreased by the addition of 4% ethanol and 0.1% peracetic acid in pure water for 20 hours with stirring. The tissue fragments were washed for another 48 hours in pure water and then lyophilized for 3 days. The decellularized porcine corneas were kept at −80° C. for no more than 3 months.

Cryo-Pulverization and Digestion of Decellularized Porcine Corneas. The lyophilized and decellularized porcine cornea tissue pieces were cryo-pulverized using a Spex 6700 freezer-mill. The pulverized tissue was then digested with pepsin/HCl at a ratio of 20 mg decellularized extracellular matrix (ECM) to 1 mg>400 U pepsin in 0.1 M HCl for 72 hours at room temperature. At the end of digestion, when tissue particles were no longer detectable, the solution was neutralized to pH 7.5 with NaOH and 10×PBS. This provided a thermoresponsive hydrogel, referred to herein as a COMatrix hydrogel, which has thermo-gelation properties at 37° C. The COMatrix hydrogel was used as a control in experiments described herein.

Functionalization of Decellularized and Digested Porcine Corneas. The thermoresponsive COMatrix hydrogel was reacted with methacrylate anhydride (MA) with different w/w ratios (2:1, “0.5×;” 1:1, “1×;” and 1:2, “2×”). The MA was added dropwise and the pH was adjusted to 7.5. The reaction was carried out at 4° C. for 12 hours. The solution was diluted five times with PBS and dialyzed against deionized water for 4 days using 12-14 MWCo dialysis tubes (Sigma). At the end of dialysis, the functionalized COMatrix hydrogels were snap-frozen in liquid nitrogen and lyophilized for 3 days.

Fabrication of Light-Curable COMatrix Hydrogel. To formulate the light-curable COMatrix hydrogel, a visible light photoinitiating system was used. The photoinitiating cocktail was prepared by dissolving Eosin Y (0.1 mM final concentration), triethanolamine (TEOA, 0.75% w/v final concentration) and N-vinylcaprolactam (VC, 0.5% w/v final concentration) in 1×PBS. The functionalized COMatrix hydrogels prepared with different ratios of MA (i.e., 0.5×, 1×, and 2×) were then dissolved in the photoinitiating cocktail (25 mg/ml) to make the ready-to-cure LC-COMatrix hydrogel. The light-curable LC-COMatrix hydrogels were then loaded in 1 ml syringes with luer locks and stored at 4° C. for future use. To apply the light-curable LC-COMatrix hydrogels, a 22-gauge angled cannula was used. Light-curing was performed using a custom made light source fabricated from green LEDs (520 nm, 30° Beam angle) with emission of 100 μW/cm². All light-curing was performed for 4 minutes unless otherwise specified.

Characterization of Light-Curable COMatrix Hydrogels. The collagen and sulfated glycosaminoglycans (sGAGs) content of the fabricated light-curable LC-COMatrix hydrogels, thermoresponsive COMatrix hydrogels, and human corneas (Eversight Eye Bank, Illinois) were measured using conventional methods.

Measuring the Degree of Functionalization. To confirm the derivatization of the methacrylate groups and to measure the degree of functionalization (DoF) in fabricated 0.5×, 1× and 2× LC-COMatrix hydrogels, 1H NMR (Nuclear magnetic resonance) and fluoraldehyde assay were used in accordance with known methods.

To measure the DoF of 0.5×, 1× and 2× LC-COMatrix hydrogels using the fluoraldehyde assay, the LC-COMatrix hydrogels from the same batch were used to draw the standard curve. In brief, the LC-COMatrix hydrogels were dissolved in PBS (0.5 mg/ml) and the same batch of COMatrix hydrogel were dissolved in PBS (0.05, 0.1, 0.5, 1 and 2 mg/ml). Subsequently, 300 μl of each sample and PBS (control) were mixed with 600 μl of room temperature fluoraldehyde reagent (Sigma) for 1 minute. After that, 250 μl of each mixture was loaded in triplicate in an opaque 96-well plate and the fluorescence intensities were measured at 450 nm with excitation at 360 nm. The average intensity of PBS was deducted from the average intensity of LC-COMatrix hydrogel and COMatrix hydrogel (standard) samples. The linear calibration curve was drawn using the COMatrix hydrogel fluorescence intensities and the DoFs for 0.5×, 1× and 2× LC-COMatrix hydrogels were calculated using the standard curve.

Dry Weight and Swelling Behavior of Light-Curable COMatrix Hydrogel. Eighty μl of COMatrix hydrogel or 0.5×, 1× and 2× LC-COMatrix hydrogel were loaded in a 7-mm diameter PTFE ring (520 μm thickness). The COMatrix hydrogel was incubated at 37° C. for 30 minutes, and the LC-COMatrix hydrogels were cured with green light for 4 minutes. An equivalent amount of fibrin glue (TISSEEL Fibrin Sealant) was loaded in the ring and incubated at 37° C. for 30 minutes for full reaction. Cadaveric human corneas (Eversight Eye Bank, Illinois) with an average thickness of 500-550 μm were also cut with a 7-mm diameter trephine. All samples were washed briefly with PBS, weighted (W0), placed in 500 μl PBS, and incubated at 37° C. The weight of samples were followed at days 1 (W1), 7 (W7) and 18 (W18) after removing residual solution on the surface. The dry weight (DW) of all samples was determined after the samples were fully dried in an oven at 90° C. for 3 hours. The water content (%) of each sample (N=3) at each day was then calculated using the weight at each day of follow-up (Wd) according to the following equation: Water Content (%)=Wd−DW/Wd×100. The change in water content each day was then plotted to map the swelling behavior.

Enzymatic Degradation of LC-COMatrix Hydrogels. To track collagenase degradation of LC-COMatrix hydrogels compared to COMatrix hydrogels, fibrin glue and human cornea, each sample (N=3) was incubated in 200 μl of collagenase type 1 (Sigma, 2 μg/ml in 50 mM TES and 0.36 mM CaCl). Each sample was weighted on days 0, 1, 2, 5 and 10 and the weight change (%) was calculated.

Mechanical Characterization of LC-COMatrix Hydrogels. Compression testing of cylindrical-molded LC-COMatrix hydrogels with 8 mm diameter and 1.8 mm height (N=3) was performed according to known methods. In brief, a mechanical testing machine (2251b Actuator, Test Resources) equipped with a 5 N load cell was used with a constant crosshead speed of 1%/sec until reaching the 50% compressive strain. The strain (%)-stress (kPa) curve was plotted using the recorded data.

Viscosity Measurement of LC-COMatrix Hydrogels. To measure the viscosity of the 2× LC-COMatrix hydrogel, the hydrogel was loaded in a rotational rheometer (Kinexus Ultra+, Malvern) with a parallel 25-mm plate and temperature controller. The gap was set to 0.4 mm and the viscosity (Pas) was recorded with shear rate change from 0.01 to 1000 (S⁻¹) at different temperatures (12, 25 and 37° C.). The same recording setting was used for fibrin glue (TISSEEL, Baxter) and DISCOVISC® viscoelastic (Alcon Laboratories, Inc). The recorded viscosities were plotted against shear rate.

Rheological Photo-Gelation Kinetics of LC-COMatrix Hydrogels. The 0.5×, 1× and 2× LC-COMatrix hydrogels were loaded on a quartz bed of a rotational rheometer (Kinexus Ultra+, Malvern) with a parallel 25-mm plate and a custom-made green light source. The gap was adjusted to 0.4 mm and the shear moduli (elastic, G′ and viscous, G″) were recorded with a frequency of 0.159 Hz and strain of 5%. After one minute of recording, the green light was turned on while recording continued and the light was turned off at minute 5. The recording was continued for another 5 minutes. Subsequently, the rheological properties of photogelated LC-COMatrix hydrogels were evaluated with strain sweep (0.1 to 100% at 0.159 Hz frequency) and frequency sweep (0.1 to 100 Hz at 5% strain). All the above tests were also performed on fibrin glue prepared per manufacturer's instructions and incubated for 30 minutes for full reaction.

Ex Vivo Bio-Adhesion Strength Measurements. The bio-adhesion strength of 0.5×, 1× and 2× LC-COMatrix hydrogels was measured using cadaveric human corneas as substrate. The human corneas (average thickness of 500 μm) were cut using 10 mm trephine and then cut in half. Each half was loaded on an already half-cut contact lens holder to preserve the cornea curvature. Two mm of half-corneas was in touch with a half-contact lens holder secured with cyanoacrylate glue (Krazy Glue, Elmer's Products Inc.). The contact lens holder was secured in grips of a mechanical testing machine (2251b Actuator equipped with a 5 N load cell, Test Resources) and aligned with 1-mm gap. The LC-COMatrix hydrogels were loaded in between the half-corneas with a length of 3 mm and thickness of 0.5 mm using the 22-gauge angled cannula. The LC-COMatrix hydrogels were then cured with a custom made green light source for 4 minutes. The tensile test was run at a rate of 1 mm/min and the adhesion strength (MPa) was calculated using the highest recorded load (N) divided by the surface area (3 mm×0.5 mm)).

Ex Vivo Burst Pressure Measurements. The ex vivo sealing capability of the 0.5×, 1× and 2× LC-COMatrix hydrogels was evaluated using a burst pressure measurement setup. The cadaveric human corneas were loaded in an artificial anterior chamber (CORONET®) connected to a same-level pressure sensor and pump. Four different sizes/types of controlled corneal injuries were created on the cadaveric human corneas (at pressure of 18 mmHg) including 1 mm and 2 mm round full-thickness injury made using a dermal punch (where the cut tissue was removed to create a hole); and, 2.75 mm and 5.9 mm cut (laceration) injuries made with 2.75 mm keratome and scalpel (size 11) knives, respectively. After removing the air from the artificial anterior chamber and drying the area of injury, the LC-COMatrix hydrogels were applied using a 22-gauge cannula and cured with green light for 4 minutes. The pressure was increased by controlled injection of PBS (dyed blue, loaded in a 20 ml syringe) at a speed of 1 ml/min until burst/failure was visualized in the video recording viewed from above. 2× LC-COMatrix hydrogels showed the best performance in the characterization and ex vivo experiments. Thus, this matrix was used for further in vitro, ex vivo and in vivo experiments.

Harvesting Human Corneal Epithelial and Stromal Cells. Immortalized human corneal epithelial cells (HCECs) were expanded in high-glucose DMEM medium (4500 mg/L, Fisher Scientific) containing 10% Fetal Bovine Serum (FBS) (Fisher Scientific) and 1× Antibiotic-Antimycotic (Fisher Scientific) for no more than 40 passages. The human corneal MSCs (hcMSCs) were obtained from cadaveric human corneas as previously described (Yazdanpanah et al. (2021) Tissue Eng. Part C Methods 27 (5):307-321). In brief, the center of cadaveric human corneas (Eversight Eye Bank, Illinois) were cut with 8 mm trephine and the periphery (containing the limbal area) was cut into 4 pieces. Each piece was then put in a well of 6-well plate until hcMSCs outgrowth was observed. The outgrown cells (P0) were collected and expanded. hcMSCs with passage 3 and 4 were used for further experiments. For culture media, α-MEM medium (Fisher Scientific) containing 10% FBS (Fisher Scientific) and 1× Antibiotic-Antimycotic (Fisher Scientific) was used in all experiments with hcMSCs.

Two-Dimensional and Three-Dimensional Cull Cultures. The cell compatibility of 2× LC-COMatrix hydrogels as compared to thermoresponsive COMatrix hydrogels was evaluated. The cell-free thermogelation was induced as described herein by incubating the tissue culture plate at 37° C. for 30 minutes (75 μl COMatrix hydrogel, 25 mg/ml, was loaded in each well of a 48-well plate, N=3). The cell-free photogelation of 2× LC-COMatrix hydrogels was induced by curing the hydrogel loaded in a 48-well plate (25 mg/ml, 75 μl in each well) with green light for 4 minutes. Subsequently, the HCECs or hcMSCs (3×10³ cells) were seeded on top of the gelled COMatrix hydrogel using the above-mentioned media for each cell type (2D cell culture).

For 3D cell culture, the hcMSCs (3×10⁴ cells) were mixed with 80 μl of 15 mg/ml 2× LC-COMatrix hydrogel, loaded in a well of a 48-well plate, and cured with green light for 2 minutes. Subsequently, 300 μl of α-MEM media containing 10% FBS and 1× antibiotic was added to each well. The hydrogels were cultured for two weeks.

Live-Dead and Metabolic Activity Assays. To monitor the viability and number of two-dimensional seeded HCECs and hcMSCs on COMatrix hydrogel and LC-COMatrix hydrogel, live-dead and metabolic activity assays were performed, respectively. At days 1, 4, 9 and 15, the cells were stained with Calcein-AM (live cells), propidium iodide (PI, dead cells) and Hoechst 33342 (total cells, all from Sigma, USA) for 1 hour at 37° C. in humidified atmosphere with 5% CO₂. The cells were imaged using ZEISS Cell Observer SD Spinning Disk Confocal Microscope (Zeiss, Germany). The metabolic activity of cells as representative of cell numbers was also assessed using Cell Counting Kit-8 (CCK-8, Sigma) per the manufacturer's recommendation.

Immunofluorescence Staining. The 2× LC-COMatrix hydrogels combined with hcMSCs cultured for 2 weeks were fixed in 4% paraformaldehyde (PFA) overnight, embedded in optimal cutting temperature (O.C.T., TissueTek) and frozen on dry ice. The fixed hydrogels were sectioned by Cryostat (Fisher Scientific) and transferred onto a histological slide. The slides were then fixed with 4% PFA for 15 minutes and washed with PBS. The samples were blocked with 3% bovine serum albumin (BSA) for an hour and incubated with primary antibodies (Table 1) overnight at room temperature. The sections were subsequently washed with PBS and incubated with secondary antibodies for 1 hour at room temperature. After several rounds of washing, the slides were mounted with PROLONG™ Gold Antifade Mountant with DAPI (Thermo Fisher Scientific) and visualized with a confocal microscope (LSM 710, Carl Zeiss, Germany). The Images were analyzed with ZEN Lite software (Zeiss, Germany).

TABLE 1

| Antibody* | Source | Type | Conjugation | Dilution |
|---|---|---|---|---|
| Anti-CD90 | Mouse | Human IgG | FITC | 1:100 |
| Anti-Ki-67 | Rabbit | Human IgG | — | 1:100 |
| Anti-α-SMA | Rabbit | Human IgG | — | 1:100 |

*Fisher Scientific

Ex Vivo Retention of LC-COMatrix Hydrogel in Human Corneal Stromal Defect Model. An anterior lamellar cut (10 mm diameter, 300 μm thickness) was made in cadaveric human corneas and the anterior stromal flap was removed. The defects thus created were then repaired with 2× LC-COMatrix hydrogel (25 mg/ml), which was cured with green light for 4 minutes. The corneas (N=3) were placed in donor cornea holders containing 9 ml of Life4C (donor cornea preservative solution) and placed on an orbital shaker upside down (the corneas were face up with a layer of solution covering them). Subsequently, the shaker was placed in a 37° C. incubator and shaking was commenced at 50 orbital shakes per minute. The human corneas were then evaluated up to 30 days via slit-lamp biomicroscopy, optical computed tomography (OCT) and pachymetry to determine the adhesiveness/attachment, transparency, morphology and thickness of the corneas repaired with the cured LC-COMatrix hydrogels. As a control, human corneal stromal defects were also repaired with fibrin glue.

Rabbit Corneal Surgeries Rabbit Corneal Perforation and Stromal-Defect Models: Model Creation and Repair with LC-COMatrix Hydrogels. New Zealand rabbits were anesthetized using subcutaneous (SC) injection of Ketamine (45 mg/kg) and Xylazine (5 mg/kg), and a drop of proparacaine 0.5% was instilled into the right eye. Povidone-iodine 1% was then applied to the eye and removed after 30 seconds with a sterile sponge. A sterile drape covered the surrounding tissue of the right eye.

To create the full thickness corneal perforation model, a partial thickness lamellar keratectomy was done via a 3 mm trephination (more than half of the corneal thickness) at the center of cornea followed by performing the lamellar keratectomy using a 1.2 mm angled mini-crescent knife. Subsequently, a full thickness cut was performed at the center of the lamellar keratectomy area using a 1-mm punch biopsy and the tissue was removed using a mini-crescent knife (if needed). After drying the drained fluid from the anterior chamber, the created perforation defect was filled with 2× LC-COMatrix hydrogel, which was cured in situ with green-light for 4 minutes. After assuring that there was no leakage, a 14 mm soft contact lens was placed on the eye and remained there for at least 24 hours.

To create the partial thickness corneal stromal-defect model, an anterior lamellar keratectomy was performed as explained above. Then, a proper amount of the 2× LC-COMatrix hydrogel was applied to fill the defect and the excess was trimmed. After that, an 8 mm diameter contact lens was fit on the defect area to adjust the hydrogel with surrounding tissues, and the hydrogel was cured in situ with visible green-light for 4 minutes. A 14 mm contact lens was kept on the cornea for at least 24 hours.

The eyes from both corneal injury models were treated with an eye drop containing dexamethasone, neomycin and polymyxin B two times per day for 7 days after surgery. The follow-ups were performed with OCT imaging and pachymetry, slit-lamp biomicroscopy and fluorescein staining up to 30 days. The intraocular pressure (IOP) of the eyes (corneal perforation model) was also measured using a hand-held tonometer (Tonopen, Reichert) during the follow-ups.

Rabbit Corneal Tissue Examinations Histological Evaluations and Immunofluorescence Staining. After humanely euthanizing the rabbits with pentobarbital overdose (1 ml/10 lbs, IV) under anesthesia (ketamine/xylazine, SC), the corneas were removed and fixed overnight in 4% paraformaldehyde (PFA). Then, the tissues were washed with PBS and sequentially transferred to 15% sucrose solution in PBS and then 30% sucrose solution in PBS. Subsequently, the samples were embedded in O.C.T., frozen on dry ice, sectioned by Cryostat (Fisher Scientific), and transferred onto a histological slide for staining. Hematoxylin and eosin staining was performed in accordance with known methods. Immunofluorescence staining was performed as described herein using primary antibodies as indicated.

Compositional and Physical Characterization of LC-COMatrix Hydrogel. An improved thermoresponsive COMatrix hydrogel prepared with decellularized porcine corneal tissue was developed. The improved hydrogel composition is representative of natural corneal composition with improved characteristics such as easy handling/administration, fast in situ cross-linking, malleability, and enhanced mechanical stability. Advantageously, the improved hydrogel can be used as an in situ corneal stromal regenerative material or can be applied as a bio-adhesive for closing and repairing corneal macro-penetrations following surgeries and traumas.

By way of illustration, decellularized corneal extracellular matrix was functionalized with an acrylate, in particular methacrylate anhydride (MA), and an exemplary thermoresponsive, in situ Light-Curable Cornea Matrix (LC-COMatrix) prepared. In this example, a visible light curing system was created by combining the LC-COMatrix hydrogel with FDA approved photo-initiating cocktail composed of eosin Y, triethanolamine (TEOA) and N-vinylcaprolactam (VC). By curing the prepared combination with green light (520 nm, eosin Y has the highest absorbance rate in this wavelength), the cross-linking reaction was initiated and the viscous hydrogel was strongly stiffened. For example, 4-minute green light-curing provided an optimal cross-linked LC-COMatrix hydrogel. Advantageously, the LC-COMatrix hydrogel composition in accordance with this invention can be prepared as a ready-to-cure composition, which is loaded in syringes for further experiments and stored at 4° C. up to 4 months or longer.

Different degrees of functionalization of the COMatrix hydrogel were achieved by reacting the hydrogel with methacrylate anhydride at different ratios including 2:1 (0.5×), 1:1 (1×), and 1:2 (2×). The degree of functionalization was measured with Nuclear Magnetic Resonance (NMR) spectroscopy and fluoraldehyde assay. The NMR spectroscopy showed the peaks for methacrylate groups at 5.5 to 6 ppm compared to non-functionalized COMatrix hydrogel. As the ratio of MA to COMatrix hydrogel increased, the methacrylate peaks became stronger indicating more attached methacrylate groups to the proteins and glycan amine groups of the COMatrix hydrogel. This was consistent with results from the Fluoraldehyde assay, which measures the degree of functionalization (DoF) by quantifying the amount of free amine groups. By increasing the ratio of MA:COMatrix hydrogel from 1:2 to 1:1 and then 2:1, the number of free amine groups decreased and the DoF rose. The DoF for 0.5×, 1× and 2× LC-COMatrix hydrogels were 12.7±2.5%, 40±4.5% and 70.3±5.2% (P<0.0001, ANOVA), respectively.

To evaluate whether the functionalization process influenced the general composition of the COMatrix hydrogel, the collagen and sulfated glycosaminoglycan compositions in the COMatrix hydrogel and functionalized LC-COMatrix hydrogels were measured and compared with human cornea. The results of this analysis indicated that similar collagen and SGAG concentrations were present in all COMatrix and LC-COMatrix samples indicating that the MA functionalization process did not significantly affect the overall composition of the COMatrix hydrogel. Moreover, similar concentrations of collagen and sGAGs were observed between LC-COMatrix hydrogel samples and human corneas indicating that the LC-COMatrix hydrogel was compositionally representative of human cornea.

Water content and swelling behavior of the LC-COMatrix hydrogels, the 0.5×, 1× and 2× LC-COMatrix hydrogels (25 mg/ml) were also determined by loading the hydrogels in 7-mm diameter PTFE rings with a thickness of 550 μm and curing with green light for 4 minutes. The thermoresponsive COMatrix hydrogel (25 mg/ml) and fibrin glue (TISSEEL Fibrin Sealant) were also loaded in the same ring and incubated at 37° C. for 30 minutes. Human corneas (average thickness of 500-550 μm) were also cut with a 7-mm diameter punch biopsy. All the samples (N=3 per each sample) were incubated in the same amount of PBS solution at 37° C. and the weight of each sample was determined for 18 days. At the end, all the samples were fully dried in an oven and their dried weights were measured to calculate the water content of each sample. The water content of the COMatrix and LC-COMatrix hydrogels was more than 95%, and was not significantly different than the fibrin glue. In comparison, the human cornea had a lower water content (85.7±1.5%). It was observed that the human cornea buttons experienced a primary swelling of 3.3±0.9% while the 2× LC-COMatrix had less than one percent change in water content for the first day. The change in the water content between day 1 and day 18 was negligible for each of the COMatrix hydrogel, LC-COMatrix hydrogels and human cornea indicating no significant swelling. Interestingly, the fibrin glue steadily lost water weight during this experiment.

The effect of collagenase on the thermogelled COMatrix hydrogel (25 mg/ml) and photogelated LC-COMatrix hydrogels (25 mg/ml, 4 minute curing) were compared to fibrin glue and human cornea by weighing the samples during the experiment. The human corneas experienced a primary increase in weight, which indicated swelling. Subsequently, the human corneas decreased in size by 68.8±5.3% over the 10 days of treatment with collagenase. The decrease in size of the LC-COMatrix hydrogels was correlated with their DoF; the 0.5× and 1× LC-COMatrix hydrogels were 100% degraded after 10 days while the 2× LC-COMatrix hydrogel was reduced in size by 90.4±1.7%. The COMatrix and fibrin glue were also 100% degraded after 10 days; however, the degradation rates were faster.

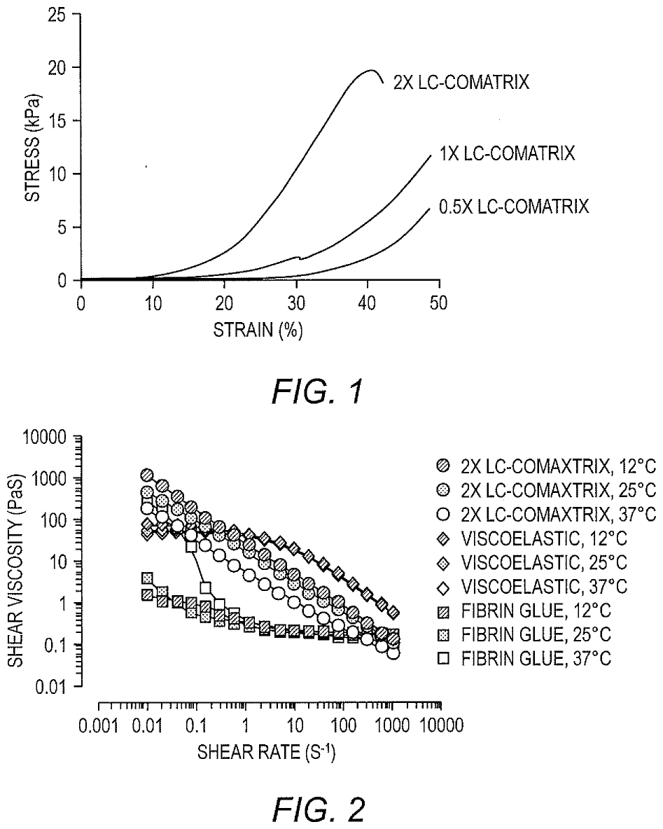

Upon visual inspection, an 8-mm diameter and 1-mm thick green light cured 2× LC-COMatrix hydrogel exhibited excellent structural integrity and a smooth surface. The LC-COMatrix hydrogel disks underwent compressive tests up to 50% strain. Each of the 0.5×, 1× and 2× LC-COMatrix hydrogels showed an increase in stress following a rise in the strain (FIG. 1). The increase in stress was correlated with DoF of the LC-COMatrix hydrogels with the 2× LC-COMatrix hydrogel reaching 20 kPa stress at near 40% strain (FIG. 1).

Rheological Characterization and Photogelation Kinetics of LC-COMatrix Hydrogels. One of the main challenges for the application of bio-adhesives in the field of ophthalmology is the consistency of the bio-adhesive in the applied area. The bio-adhesive not only needs to have strong adhesion, especially after polymerization, but also it is crucial to have robust cohesion even before cross-linking. If the bio-adhesive has low cohesion (very liquid) it will spread across the field to the undesired areas before cross-linking. Conversely, if it is too viscous (very high cohesion), it will not provide adequate coverage over the desired area. For example, fibrin glue and cyanoacrylate on the corneal surface exhibit low cohesion, which leads to spreading on the surface to the unwanted areas after application and before cross-linking. Likewise, Gelatin methacrylate (GelMA) has the same drawback; at room or lower temperatures GelMA has a very strong cohesion, however, after warming to 37° C. the cohesion is decreased significantly resulting in dispersal to undesired areas. One of the most common materials used in ophthalmology is a combination of sodium hyaluronate and chondroitin sulfate. This “viscoelastic” material has suitable cohesion that provides augmentation and protection in ophthalmic surgeries.

Therefore, the rheological characteristics of the 2× LC-COMatrix hydrogel were determined and compared to viscoelastic and fibrin glue precursors. Viscosity measurements were taken at 12, 25, and 37° C. at increasing shear rates from 0.01 to 1000 (S⁻¹). This analysis indicated that the viscosity of 2× LC-COMatrix hydrogel was considerably higher than fibrin glue and had minimal dependence on temperature (FIG. 2). Thus, the 2× LC-COMatrix hydrogel does not spread to the undesired areas when applied. Also, the 2× LC-COMatrix hydrogel exhibited shear thinning similar to the viscoelastic material. This characteristic makes the LC-COMatrix hydrogel suitable for administration by injection as well as applications such as 3D-printing.

To record the photocuring kinetics by rheometry, the 0.5×, 1× and 2× LC-COMatrix hydrogels were loaded on a quartz glass surface of the rheometer with a green light source (100μW/cm²) installed at the bottom. The gap junction was set to 0.4 mm and the shear moduli (elastic, G′; and viscous, G″) were recorded at 0.159 Hz frequency and 5% strain for 1 minute. The green light was turned on for 4 minutes while the recording continued. The LC-COMatrix hydrogels rapidly hardened in the first minute of curing with green light, which became more flattened and plateaued depending on the DoF. For 0.5× LC-COMatrix hydrogel, the cross-linking finished after almost 1 minute and the elastic modulus did not increase. In the case of 1× LC-COMatrix and 2× LC-COMatrix hydrogels, more cross-linking occurred after the first minute surge evident by an increase in G′ with the slope of cross-linking consistent with the DoF. To measure the shear moduli of the fibrin glue, fibrin glue precursor was loaded on the rheometry plate and incubated for 30 minutes while recording with the same frequency and strain as the LC-COMatrix hydrogels. The average G′ and G″ for photocured 2× LC-COMatrix hydrogel was 7797±546 Pa and 250±39 Pa, respectively, which was higher than fibrin glue with G′ of 4722±346 Pa and G″ of 584±76 Pa (N=3). The 1× LC-COMatrix and 0.5× LC-COMatrix hydrogels had average G′ of 2670±534 Pa and 424±38 Pa, and average G″ of 163±26 Pa and 29±7 Pa, respectively. The rheological characteristics of cured LC-COMatrix hydrogels with different DoFs were evaluated with frequency sweep and strain sweep and compared with fibrin glue. The strain sweep from 0.1 to 100% showed that the linear viscoelastic (LVE) behavior of cured LC-COMatrix hydrogels as below 10%. Therefore, the 0.5% strain (in the LVE range) was used to perform the frequency sweep from 0.159 Hz to 100 Hz. The behavior of cured LC-COMatrix hydrogels was similar to fibrin glue and GelMA.

Ex vivo Adhesion Strength of LC-COMatrix Hydrogels on Human Corneal Substrates. Tensile adhesion tests using human corneas as the substrate were conducted to measure the adhesion strength of the LC-COMatrix hydrogels. Human cadaver corneas (Eversight Eye Bank, Illinois) used in this analysis had been preserved in a storage solution (Life4C) for no more than 14 days. The human corneas (average 550 μm thickness) were first cut with a 10-mm diameter trephine and then cut into half. Subsequently, each half was loaded on a contact lens holder (2-mm from the edge) and secured with cyanoacrylate. The contact lens holders were fixed in the tensile testing machine grips. A 1-mm distance was set between the half corneas. The 0.5×, 1× and 2× LC-COMatrix hydrogels and fibrin glue (N=6) were loaded in the middle of the two cornea pieces (3-mm length) and cured with green light for 4 minutes. At the end, the tensile test was performed with 1 mm/min speed and the highest load was recorded. The highest load divided by the surface areas is equal to adhesion strength of the sample. The adhesion strength of the cured 2× LC-COMatrix hydrogel was 21.8±2.3 kPa which was significantly higher than that of fibrin glue (4.9±2.3 kPa, p<0.0001). The adhesion strengths of 1× and 0.5× LC-COMatrix hydrogels were 11.1±3.7 kPa and 3.6±1.8 kPa, respectively.

Ex vivo Closure of Penetrating Injuries in Human Corneas with LC-COMatrix Hydrogels. To assess the utility of the LC-COMatrix hydrogels in wound repair, burst pressure measurements were taken. Cadaveric human corneas were loaded in an artificial anterior chamber connected to a pressure sensor and a pump. After assuring a sealed system, the pressure behind the human corneas were adjusted to 18 mmHg and the following injuries were created: 2 mm and 1 mm full thickness punch injuries (the cut tissue was removed), and 2.75 mm and 5.9 mm full thickness stab injuries created by surgical knives. After drying the injury sites, 0.5×, 1× and 2× LC-COMatrix hydrogels were applied on the injury site and cured in situ with green light for 4 minutes. The pressure was increased slowly until the burst pressure was reached while recording with a video camera. Burst pressures (BP) for the LC-COMatrix hydrogels with various DoFs were measured and compared to fibrin glue (N=4).

The average burst pressure of the 2 mm punch injury repaired with 2× LC-COMatrix hydrogel was 327±175 mmHg, while the burst pressure for fibrin glue was 11±4 mmHg (p<0.0001, D). The 0.5× and 1× LC-COMatrix hydrogels had weaker potential to keep the 2 mm punch injury sealed and the burst happened at lower pressures (1× LC-COMatrix hydrogel BP, 240±113 mmHg; 0.5× LC-COMatrix hydrogel BP, 151±71 mmHg), however, these burst pressures far exceeded that of the fibrin glue. The same pattern was repeated for other types of corneal penetration injuries as well; the burst pressures of 1 mm punch injury, 2.75 mm cut injury, and 5.9 cut injury repaired with 2× LC-COMatrix hydrogel were 300±160 mmHg, 542±86 mmHg, and 188±63 mmHg, respectively, whereas closure with fibrin glue provided burst pressures or 13±2 mmHg, 19±4 mmHg, and 8±4 mmHg, respectively (p<0.001 for all comparisons). Again, while the 0.5 and 1× LC-COMatrix hydrogels exhibited lower burst pressures than the 2× LC-COMatrix hydrogel for the 1 mm punch injury, 2.75 mm cut injury, and 5.9 cut injury, the burst pressures for 0.5 and 1× LC-COMatrix hydrogels in these models far exceeded that of the fibrin glue. For subsequent in vitro, ex vivo and in vivo experiments, 2× LC-COMatrix hydrogels were primarily studied.

In vitro Cytocompatibility of LC-COMatrix Hydrogel. Two of the most abundant cell types in the cornea are epithelial cells that cover the cornea surface, and the stromal cells residing in corneal stroma. Any biomaterial to be applied for repair of the corneal stroma should be compatible with the proliferation of these cell types. Corneal epithelial cells will grow on the surface while corneal stromal cells will migrate into the applied biomaterial to repair the stromal defect over time. To assess the biocompatibility of 2× LC-COMatrix hydrogel and compare the results with thermoresponsive COMatrix, the light-curable and thermoresponsive hydrogels (25 mg/ml) were loaded in 96-well plates and cured with green light (4 minutes) or incubated at 37° C. (30 minutes), respectively. Human corneal epithelial cells (HCECs) and human corneal mesenchymal stem cells (hcMSCs) derived from cadaveric human corneas were seeded on the cured LC-COMatrix hydrogel and thermogelated COMatrix. The viability and proliferation of cells were followed by live-dead assay.

The results of this analysis indicated that both HCECs and hcMSCs exhibited more than 95% viability at follow-up and proliferation was observed. Moreover, the number of viable cells were tracked using a non-toxic metabolic assay (CCK-8 assay) at days 1, 4, 9 and 15. Both HCECs and hcMSCs experienced a lag growth phase until day 9 after which the cells continued growing steadily from day 9 to day 15 on both COMatrix and LC-COMatrix hydrogel.

One of the main concerns regarding the interaction of a biomaterial and hcMSCs is the transdifferentiation of hcMSC to smooth muscle cells. Smooth muscle cells in the cornea are the main mediator of corneal scarring leading to corneal opacity. To evaluate whether the LC-COMatrix hydrogel induced transdifferentiation of hcMSC to smooth muscle cells, the LC-COMatrix hydrogel (15 mg/ml) was combined with hcMSCs, the mixture was exposed to green light (2 minute) to cure the hydrogel, and the cross-linked hydrogel (containing the cells inside) was cultured for 2 weeks. After two weeks, the hydrogel disks were stained for the expression of CD90 (a marker of hcMSCs), Ki-67 (an indicator of cell proliferation), and α-SMA (a marker of smooth muscle cells). Ki-67 marker was strongly expressed in the CD90 positive hcMSCs indicating active cell proliferation. On the other hand, no expression of α-SMA was detected in the CD90 positive hcMSCs, indicating that the human corneal mesenchymal stem cells did not transdifferentiate into smooth muscle cells when seeded in LC-COMatrix hydrogel.

Ex vivo Retention of LC-COMatrix Hydrogel Applied to Repair the Human Corneal Stromal Defect. To explore the retention of LC-COMatrix hydrogel in cases of corneal stromal defect repair, cadaveric human corneas with stromal defects were used. For this analysis, an anterior lamellar cut with diameter of 10 mm and thickness of 300 μm was made in cadaveric human corneas and the anterior flap was removed. Afterwards, the created corneal stromal defect was repaired with 2× LC-COMatrix hydrogel and cured with green light for 4 minutes. The repaired corneas were then put anterior down in the eye bank cornea-holders containing 9 ml Life4C solution. The cornea-holder was placed upside down on a rotational shaker and rotated at a speed of 50 cycles/min at 37° C. for a month. The repaired corneas were analyzed via slit lamp biomicroscopy, optical coherence tomography (OCT) and pachymetry to track the transparency, structure and thickness map of the repaired human corneas, respectively. This analysis indicated that the LC-COMatrix hydrogel consistently repaired the human corneal stromal defect with a smooth surface having comparable transparency to native human corneas. During the 30 days of incubation, the repaired corneas were under constant flow of corneal preservative solution and no sign of detachment was observed on OCT imaging. Moreover, the thickness of repaired corneas remained stable for 30 days as evident by the results of pachymetry.

In vivo Closing of Macro-Perforation in Rabbit Cornea using LC-COMatrix Hydrogel. A rabbit corneal macro-perforation model was created to assess the potential of LC-COMatrix hydrogel to close/repair corneal penetrations. A partial thickness of the rabbit cornea was removed by a 3-mm diameter lamellar keratectomy. Then, a full-thickness cut was made in the center of the created lamellar stromal defect using a 1-mm trephine. The anterior chamber was totally flat due to drainage of fluid after creating the perforation model. The iris was attached to the cornea and the lens was also touching the cornea. However, after closing the corneal perforation with LC-COMatrix hydrogel, the anterior chamber became deep at follow-up. During a perforation, a part of the iris will attach to the repaired area. In two rabbits, the iris was fully detached and in the other rabbit the iris remained partially attached until the last follow-up. The corneal epithelium grew back on the defect area in about a week as no fluorescein staining was observed at day 14 of follow-up. The intraocular (IOP) pressure in the surgical eyes were not significantly different from the control eyes during the follow-up. There was some reduced transparency at the closed/repaired areas, which was due to invasion of blood vessels from the attached iris leading to faster than desired tissue remodeling. While conventional materials used in such closures require several repairs, the LC-COMatrix hydrogel was applied once and no secondary repair was performed thereafter.

In vivo Repairing of on Rabbit Corneal Lamellar Defect by LC-COMatrix Hydrogels. A rabbit corneal stromal defect was created with a 3-mm lamellar keratectomy technique. A partial thickness stromal defect was then repaired with LC-COMatrix hydrogel. LC-COMatrix hydrogel was applied to the defect, a contact lens was then used to adjust the hydrogel level with surrounding tissues, and the bio-adhesive was cured with green light for 4 minutes. Slit lamp biomicroscopy and fluorescent staining showed rapid recovery and closure of corneal epithelium over the applied LC-COMatrix hydrogel as well as prominent transparency of the repaired area during the 28 days follow-up. OCT imaging during follow-up showed substantial repair of the corneal stroma by the LC-COMatrix hydrogel.

### Example 2: In Situ Porcine Corneal Matrix Hydrogel as Ocular Surface Bandage

Bioactive substrates can be used therapeutically to enhance wound healing. Here, the effect of an exemplary in situ thermoresponsive hydrogel from suitable eye tissue sources, such as human eye tissue or decellularized porcine cornea ECM (COMatrix) was evaluated for application as an ocular surface bandage for corneal epithelial defects.

Fabrication of COMatrix Ocular Bandage Hydrogel. Under sterile conditions, porcine corneas (PCs) were dissected and washed with PBS (1×) containing 1% gentamicin, 1% penicillin and 1% streptomycin. The PCs were cut into pieces with an average size of 2×2 mm². The tissue pieces were first stirred in 20 mM ammonium hydroxide solution (Sigma) containing 0.5% X-100 TRITON (Fischer Scientific) in distilled water for 4 hours for decellularization. Tissues were then transferred to 10 mM Tris-HCl (pH 8.4, Sigma) containing 0.5% EDTA (Fisher Scientific) in distilled water and stirred for 24 hours at room temperature. The porcine cornea pieces were then stirred in 10 mM Tris-HCl containing 1% (v/v) TRITON X-100 for 24 hours at 37° C. To remove the DNA remnants, the tissue fragments were agitated in 50 mM Tris-HCl containing 7.5 U/ml deoxyribonuclease (Sigma) in molecular biology grade water (Fisher Scientific) for 16 hours at 37° C. To further remove cell remnants and chemicals, the samples were stirred in PBS for 48 hours while changing the PBS twice per day. The bio-burden of decellularized tissue pieces was reduced by stirring in 0.1% peracetic acid (32 wt % in dilute acetic acid, Sigma) in 4% ethanol in molecular biology grade water for 16 hours. Following stirring the tissues in molecular biology grade water three-times for 2hours, the tissues were snap-frozen in liquid nitrogen (30 minutes) and lyophilized for 48 hours at −55° C. and <0.133 mBar. The lyophilized tissues were than stored at −80° C. until further experiments for no more than 6 months.

To fabricate an exemplary COMatrix hydrogel from decellularized PC ECM, lyophilized tissue pieces were cryo-milled using freezer-mill (Spex 6700). The resultant fine powder was sieved using a mesh (size 40, Sigma) and partially digested by slow stirring in 0.01 M HCl (20 mg/ml) containing 1 mg/ml pepsin (>400 U/mg, Sigma) for 72 hours at room temperature. To form a hydrogel, the digested PC ECM was neutralized to pH 7 using one-ninth 0.1 M NaOH and one-tenth PBS 10×, while on the ice. The hydrogel was diluted to the desired concentrations using PBS. To induce gelation, the cool COMatrix hydrogel was incubated at 37° C. for 10 to 15 minutes. The gel formed following incubation at 37° C. and it was immediately used for further applications. Assessments showed that if the COMatrix gel formed at 37° C. was cooled down to 4° C., no change would occur to the stiffened structure of the gel. The COMatrix gel formed at 37° C. was stable in PBS incubating at 37° C. for at least 3 months.

In vitro Experiments Using Human Corneal Epithelial Cells (HCECs). Immortalized human corneal epithelial cells (HCECs) were expanded in high-glucose DMEM medium (4500 mg/L, Fisher Scientific) containing 10% Fetal Bovine Serum (Fisher Scientific) and 1× Antibiotic-Antimycotic (Fisher Scientific) for no more than 40 passages. HCECs were detached with TrypLE™ express enzyme (Fisher Scientific) for further experiments. To evaluate the interaction of HCECs with COMatrix hydrogel, in vitro assays including an attachment assay, proliferation assay and Live-Dead assay were performed. All experiments were conducted in triplicate.

Attachment Assay. To compare the attachment of HCECs to COMatrix hydrogel and plastic cell culture dish (control), 48-well plates were coated with COMatrix hydrogel (1 mg/ml, 100 μl per well) or PBS and incubated for 2 hours at 37° C. The supernatant was removed and 1×10⁴ HCECs in 200 μl complete media (High-Glucose DMEM containing 10% FBS and 1× Antibiotic-Antimycotic) were seeded in each well (6 wells per group). The plates were incubated at 37° C. for different time periods including 10, 30, 60, 120 and 240 minutes. After that, each plate was gently washed with PBS to remove the unattached cells. To compare the number of remaining attached live cells after each time period, a metabolic activity assay was performed using the Cell Counting Kit-8 (CCK-8, Sigma). Following the manufacturer's recommendation, 10 μl of the provided solution (WST-8) was added to each well containing 100 μl of complete media, after which the plate was incubated in humidified atmosphere with 5% CO₂ at 37° C. for 2 hours and the optical density (OD) was measured at 450 nm representing the number of cells in each well.

Proliferation and Live-Dead Assays. To evaluate the effect of the exemplary COMatrix hydrogel on HCECs' proliferation, two different assays were used. To measure the effect of soluble COMatrix, the cell culture media (high-glucose DMEM containing 1× Antibiotic- and no FBS) was supplemented with COMatrix (0.5 mg/ml), and HCECs were plated on the medium at a density of 2×10³ cells/well in a 96-well plate and cultured for 5 days. PBS supplementation was used as control (6 wells per group). As above, the number of live cells was measured using CCK-8.

In the second protocol, the viability of HCECs (2×10³ cells/well, 6 wells per group) cultured on COMatrix hydrogel (400 μM thickness) over a period of 1, 4, 9 and 15 days was measured by staining with Calcein-AM (live cells), propidium iodide (PI, dead cells) and Hoechst 33342 (total cells, all from Sigma) for 1 hour by incubating at 37° C. in humidified atmosphere with 5% CO₂. The HCECs were imaged using ZEISS Cell Observer SD Spinning Disk Confocal Microscope (Zeiss, Germany), and the images were analyzed using ZEN Lite software (Zeiss, Germany). Also, the number of live cells for each time point was compared to control using CCK-8 metabolic assay kit as described above.

Inflammatory Assay. To induce an inflammatory response in cultivated HCECs, a previously developed method was employed. The cells were treated with the Toll-like receptor (TLR)-3 ligand polyinosinic-polycytidylic (Poly(I)C, 100 μg/mL dissolved in DMEM medium containing 5% FBS, Sigma) with COMatrix hydrogel (0.5 mg/ml) or PBS for 24hours. After treatment, the cells were removed from each well by scraping, and the supernatants were collected and centrifuged. The pellets were then lysed with RNA lysis buffer for RNA extraction. The RNA was extracted according to the protocol described previously per manufacturer's instructions (RNeasy Protect Mini Kit, Qiagen). Reverse-transcriptase (RT) reaction was performed using extracted mRNA and a cDNA synthesis kit (SUPERSCRIPT First-Strand Synthesis System, Invitrogen).

Relative quantitative polymerase chain reaction (qPCR) was performed using intron spanning primers for human glyceraldehyde-3-phosphate dehydrogenase (GAPDH) tumor necrosis factor-alpha (TNF-α), Interleukin-6 (IL-6) and IL-1β according to the manufacturer's protocol (Fast SYBER Green Master Mix, Applied Biosystems). All samples were run in triplicates using QuantStudio 7 Flex Real-Time PCR System (ABI), and each experiment was repeated three times. Negative controls using samples without reverse transcriptase were included in the qPCR step to confirm that the results were not affected by DNA contaminants. Quantified results of each reaction were analyzed. The relative ΔΔCTmRNA expression was measured by normalization to GAPDH.

In-vivo Corneal Epithelial Wound Healing. The effects of the fabricated COMatrix ocular bandage hydrogel on corneal epithelial wound healing was evaluated using a murine 2-mm corneal epithelial debridement model. Male C57BL/6J mice (4-5 months-old) were anesthetized with intraperitoneal injection of ketamine (100 mg/kg) and xylazine (5 mg/kg). Anesthetized mice were positioned under a surgical microscope, one drop of 0.5% proparacaine was applied to the eye, and a 2-mm area was demarcated using a 2-mm trephine.

The corneal epithelial layer in the demarcated area was removed gently using an AlgerBrush II (The Alger Companies). After taking a baseline photograph of the fluorescein-stained eye using a Nikon (Tokyo, Japan) FS-2 slit-lamp biomicroscope, COMatrix ocular bandage hydrogel (15 mg/ml) was applied to the ocular surface (N=10 eyes, one eye per mouse). Then, a 4-mm-diameter transparent ACLAR® contact lens (OcuScience) was fit on the ocular surface and the COMatrix gelation was induced by applying radiation heat using a 50 Watt ceramic infrared heat emitter (KIMROO, China) for 10 minutes. The distance of the heat emitter to the mice ocular surface was adjusted to be 35 cm and the temperature was monitored every 1 minutes using an infrared thermometer with laser pointing (SOVARCATE, to keep China) the temperature no more than 37° C. on the ocular surface.

To keep the contact lens on the ocular surface, tarsorrhaphy (suturing the eyelids together) was performed according to established methods. PBS was applied in the control group before fitting the contact lens (N=10 eyes, one eye per mouse). The COMatrix ocular bandage hydrogel and overlaying contact lens were visualized by Optical Coherence Tomography (Phoenix MICRON™ Image-Guided OCT). The treated eyes were evaluated after 18 hours and photographed. The wounded area in captured photographs was measured using ImageJ software, and the ratio of wound closure relative to baseline was calculated.

Tissue Specimen Preparation and Staining. After humanely sacrificing the mice after 18 hours follow-up, the mouse eyeballs were enucleated and fixed in tissue cassettes using Optimal Cutting Temperature (Fisher Scientific) and sectioned using Cryostat (Fisher Scientific). The tissue sections were fixed 4% paraformaldehyde and then used for routine histology with hematoxylin and eosin (H&E) staining and immunostaining.

Immunostaining. The fixed tissue sections were blocked at room temperature with 3% BSA (Sigma) and incubated with primary antibodies overnight at 4° C. The primary antibodies used were specific for Ki-67 (Epitomics, 1:100) and CK-12 (Santa Cruz Biotechnology). The sections were washed and incubated with fluorescein-conjugated secondary antibodies (Jackson ImmunoResearch) for 1 hour at room temperature and counterstained with DAPI. Imaging was performed with the same light intensity and exposure for all the samples, using a confocal microscope (LSM 710, Carl Zeiss, Germany). Images were analyzed with ZEN Lite software (Zeiss, Germany).

COMatrix Hydrogel Enhances Attachment and Proliferation of HCECs and Attenuates TNF-α Expression. The fabricated COMatrix hydrogel before and after heat gel-formation and transparency its were confirmed. The bioactivity of the COMatrix hydrogel was evaluated using in vitro attachment, proliferation, and inflammatory cytokine induction assays on human corneal epithelial cells. To assess the interaction of human corneal epithelial cells with COMatrix, the attachment of cells to plates coated with hydrogel was compared to uncoated control plates. Microscopic observation of HCECs showed significantly higher attachment of HCECs on COMatrix-coated plates than control after 30 minutes. Counting the number of cells using mitochondrial metabolic assay indicated significantly faster attachment of HCECs to COMatrix compared to culture plates after 10, 30 and 60 minutes of culturing the cells.

The effect of soluble and gelled COMatrix on proliferation of HCECs was further examined in vitro. Soluble COMatrix was added (0.5 mg/ml) to the cell culture media and found to increase proliferation relative to control after 5 days (Optical Density, 1.34±0.07 vs. 1.15±0.09, P<0.001). HCECs were also seeded on COMatrix hydrogel formed at 37° C. and their metabolic activity (as a representative of cell numbers) was measured after 1, 4, 9 and 15 days. The results indicated significant enhancement of HCEC proliferation by COMatrix hydrogel as a substrate compared to tissue culture plate in all time points. Consistent with these results, HCECs seeded on COMatrix hydrogel formed at 37° C. highly expressed the proliferation Ki-67. The live-dead assay results also demonstrated high viability and a significantly higher proliferation rate of HCECs seeded on COMatrix hydrogel compared to control for 15 days.

A major pathologic mechanism prohibiting corneal repair is excessive inflammation. To study the effect of COMatrix hydrogel on inflammation, HCECs were induced with the TLR3 agonist Poly(I)C and the expression of IL-6, IL-1β and TNF-α was measured in the presence or absence of COMatrix hydrogel (0.5 mg/ml). The results showed a significant decrease in TNF-α gene expression in HCECs cultured with COMatrix compared to control cells (4.1±1.9 fold-change vs. 11.3±4.9 fold-change, respectively, P<0.05). However, no significant change was observed in the expression of IL-1Bβ and IL-6 in HCECs treated with COMatrix compared to control (2.8±0.3 foldchange vs. 2.2±0.4 fold-change for IL-1β, respectively, P>0.05; and 1.3±0.1 fold-change vs. 1.3±0.2 fold-change for IL-6, respectively, P>0.05).

COMatrix Ocular Bandage Hydrogel Enhances Corneal Epithelial Wound Closure in a Murine Model. In vivo healing effect of an exemplary COMatrix ocular bandage hydrogel was evaluated in a murine model of corneal epithelial wound healing. OCT imaging immediately after application of the COMatrix hydrogel and bandage contact lens revealed filling of the corneal epithelial defect and ocular surface coverage with COMatrix hydrogel. Observation of the COMatrix hydrogel on the murine ocular surface immediately after administration and 18 hours later indicated that remnants of the COMatrix ocular bandage were detectable on the ocular surface after 18 hours. Examination of the corneal epithelial wounds at follow-up time (18 hours) showed 71.2±13.2% wound closure in eyes treated with COMatrix in situ ocular bandage hydrogel compared to 54.7±7.8% in PBS controls (P<0.01, N=10 eyes per group).

Expression of Proliferation Marker is Significantly Higher in Corneal Epithelial Wounds Treated by COMatrix Ocular Bandage. Histologically, corneas treated with COMatrix ocular bandage hydrogel demonstrated a more intact epithelium compared to control. Immunostaining of the central cornea showed diffuse expression of CK-12 in the epithelial layer, in addition to lack of Ki-67 expression in the epithelium.

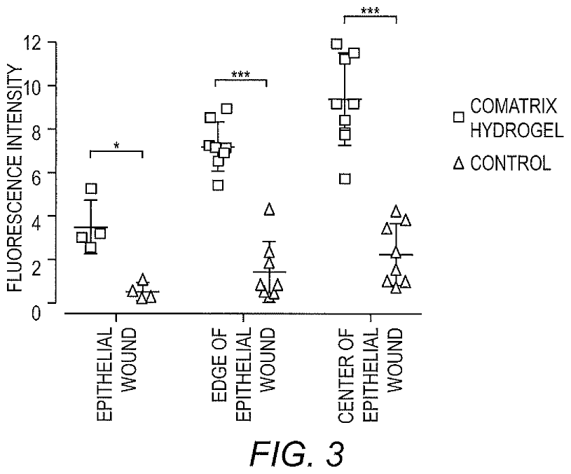

The expression of Ki-67 proliferation marker showed up-regulation of this marker at the epithelial level and under the epithelium in wounded corneas. Semi-quantitative analyses using fluorescent intensity showed a significant increase in the expression of Ki-67 at the whole corneal epithelial wound, the edge of the epithelial wound, and center of the wound in eyes treated with COMatrix ocular bandage compared to control (FIG. 3).

### Example 3: The Effect of Decellularization Protocols on Characterizations of Thermoresponsive and Light-Curable Corneal Extracellular Matrix Hydrogels

Decellularization of Porcine Corneas. Fabrication of COMatrix hydrogel required decellularization and solubilization of corneal tissues. Under sterile conditions, porcine corneas (PC) were dissected from fresh, intact porcine eyeballs that were obtained from a certified abattoir (Park Packing Co. Inc., Chicago, IL). The extracted PCs were then washed with PBS (1×) containing 1% gentamicin, 1% penicillin and 1% streptomycin. Decellularization of porcine corneas were performed using two different methods, Detergent-based (De) or Freeze-Thaw (FT). The De-based decellularization of PCS was performed as described (Yazdanpanah et al. (2021) Tissue Eng. Part C Methods 27 (5):307-321). PCs were cut into 2×2 mm² pieces; tissue pieces were initially stirred in 20 mM ammonium hydroxide solution (Sigma) containing 0.5% TRITON X-100 (Fisher Scientific) in distilled water (pH 10) for 4 hours at room temperature. Tissues were then transferred to 10 mM Tris-HCl (Sigma) containing 0.5% EDTA (Fisher Scientific) in distilled water (pH 7.5) and continuously stirred for 24 hours at room temperature. PC tissues were then continuously stirred at 37° C. for 24 hours in 10 mM Tris-HCl containing 1% (v/v) TRITON X-100 (pH 7.5).

In the FT decellularization protocol, the small pieces (2×2 mm², 20-25 corneas) of porcine corneas were put in a 50 ml tube containing 35 ml of 10 mM Tris-HCl plus protease inhibitor cocktail (complete™, EDTA-free Protease Inhibitor Cocktail, Roche) and put in a −80° C. freezer for a minimum of 5 hours. Thereafter, the tube was placed at room temperature until full thawing. The freeze-thaw cycles were repeated for 9 times. Then, the tissue pieces were washed with pure water for one day.

DNA Remnant Removal and Bio-burden Reduction. To remove DNA remnants from decellularized porcine cornea, the tissue fragments were agitated for 16 hours at 37° C., in 50 mM Tris-HCl containing 7.5 U/ml deoxyribonuclease (Sigma) in molecular biology grade water (pH 7.5, Fisher Scientific). The samples were then stirred in PBS (pH 7.5) for 48 hours at room temperature; PBS was changed twice per day. Bioburden of decellularized PC was reduced by stirring in 0.1% peracetic acid (32 wt % in dilute acetic acid, Sigma) in 4% ethanol in molecular biology grade water, at room temperature, for 20 hours. Lastly, tissues were stirred in molecular biology grade water, three times, each for two hours. Samples were assess taken at this stage to efficacy of the decellularization process (see below). Tissues were snap-frozen in liquid nitrogen for 48 hours and then lyophilized at −55° C. and <0.133 mBar. Lyophilized tissues were then stored at −80° C. until needed and for no more than 6 months. Non-decellularized corneas were lyophilized as well to be used as control (native porcine corneas).

α1-3, 6 Galactosidase Treatment. To evaluate the effects of α1-3,6 Galactosidase treatment on the α-Gal epitope removal from porcine corneas, the tissues already decellularized with FT method were treated with α1-3, 6 Galactosidase enzyme (2.5 U/ml, New England Biolabs) while incubating with DNase (see the DNAse treatment protocol above). The rest of the protocol was the same as above.

Histological Assessment. The decellularized porcine cornea tissues were evaluated by various tissue staining including hematoxylin and eosin (H&E), Alcian blue, and Picro Sirius Red staining. The decellularized tissue pieces were fixed in the TISSUE-TEK® optimum cutting temperature (O.C.T.) solution and frozen on dry ice. After sectioning using Cryostat (Fisher Scientific) and transferring to histological slides, the sections were fixed in 4% paraformaldehyde for 15 minutes and washed with deionized (DI) water. For H&E staining, the slides were incubated in hematoxylin (Fisher Scientific) for 2 minutes followed by washing with DI water. After that, the slides were briefly treated with 70% ethanol/1% HCl and washed with DI water. The slides were then stained with eosin (Fisher Scientific) for 7 minutes and then washed briefly with 95% ethanol. To evaluate the collagen distribution, Picro-sirius red staining was performed. Slides were incubated with Sirius Red (Sigma-Aldrich) in a saturated aqueous solution of picric acid for 1 hour. Then they were rinsed for one minute in 0.5% acetic acid. To visualize the sGAG content with Alcian blue staining, slides were stained with 1% Alcian Blue 8GX (Sigma-Aldrich) in 0.1M HCl for 5 minutes. Then, the slides were washed three times in deionized H₂O, each for 30-seconds. After all staining, the slides sections were dehydrated with 100% ethanol for 10 minutes followed by incubating in xylene (Fisher Scientific) for 10 minutes. After drying at room temperature, one drop of permount (Fisher Scientific) was applied to the slide and a coverslip (22×40 mm) was placed on the tissue sections and sealed with nail polish. The stained tissue sections were visualized and imaged with a light microscope (Zeiss, Germany).

Immunostaining. The tissue sections (see above for sectioning the tissues) were first fixed with 4% paraformaldehyde for 15 minutes at room temperature. They were then blocked at room temperature with 5% bovine serum albumin (Sigma). Then sections were incubated with primary antibody, mouse α-Gal Epitope (Galα1-3Galβ1-4GlcNAc-R) monoclonal IgM antibody (Enzo Life Sciences) with concentration of 1:5 and incubated at 4° C. overnight. Sections were then washed 3× with PBST (PBS+0.1% TWEEN®-20) and then incubated with goat anti-mouse IgM ALEXA FLUOR® 488-conjugated secondary antibody (Thermo Fisher) for 1 hour at room temperature. Sections were then washed with PBST 3 times and mounted with PROLONG™ Gold Antifade Mountant with DAPI (Thermo Fisher). Sections were then imaged using a confocal microscope (LSM 710, Carl Zeiss, Germany). The Images were analyzed with ZEN Lite software (Zeiss, Germany).

Thermoresponsive COMatrix Hydrogel Fabrication. To fabricate thermoresponsive COMatrix hydrogel from decellularized or native porcine corneas; first, the lyophilized tissue pieces were cryo-milled using a freezer-mill (Spex 6700). The resultant fine powder was sieved using a mesh (size 40, Sigma) and partially digested by stirring in 0.01M HCl (20 mg/ml) containing 1 mg/ml pepsin (>400 U/mg, Sigma) for 72 hours at room temperature. The digested PC-ECM was neutralized to pH 7 using one:ninth 0.1M NaOH and one:tenth PBS (10×), while on ice. The resulting hydrogel was diluted to desired concentrations using PBS (1×). To induce thermogelation, the cool COMatrix hydrogel was incubated at 37° C. for 30 to 45 minutes. The thermoresponsive COMatrix hydrogels derived from detergent-based decellularization protocol were referred to as De-COMatrix; and thermoresponsive hydrogels derived from freeze-thaw decellularization method were referred to as FT-COMatrix.

Light-Curable COMatrix Hydrogel Fabrication. To fabricate light-curable COMatrix hydrogel from the porcine corneas decellularized with detergent-based or freeze-thaw methods, the already produced thermoresponsive COMatrix hydrogels were reacted with methacrylate anhydride (Sigma) at 4° C. in the dark for 12 hours. The samples were dialyzed against deionized water for 3 days at room temperature using 12-14 kDa MWCO dialysis tubes. The functionalized COMatrix was freeze-dried for 3 days and kept in −80° C. for further experiments. To prepare the LC-COMatrix solution for experiments, the lyophilized samples were dissolved in a photo-initiating cocktail including Eosin Y, Ethanolamine, and N-Vinylcaprolactam as described herein. Afterward, the LC-COMatrix hydrogels were cured with green light (520 nm wavelength) to induce photogelation. The LC-COMatrix hydrogels derived from detergent-based decellularization protocol were referred to as De-LC-COMatrix; and light-curable hydrogels derived from freeze-thaw decellularization method were referred to as FT-LC-COMatrix.

Biochemical Quantification of COMatrix Hydrogel. Relevant assays to measure amounts of DNA, total collagen and sulfated glycosaminoglycans (sGAGs) in COMatrix hydrogel (pepsin digested decellularized ECMs) were performed. Samples were digested in papain extraction solution (1:1 v/v, 50 mL of 0.2 sodium phosphate buffer (pH 6.4), 400 mg sodium acetate, 200 mg EDTA, 40 mg cysteine HCl, 250 μl of papain suspension (Sigma) containing 5 mg of enzyme overnight at 65° C. Genomic double-strand DNA were purified using a commercially available kit (Sigma) to quantify DNA content of samples, following the manufacturer protocol. DNA concentrations were then measured using NanoDrop Microvolume Spectrophotometer (Fisher Scientific) at 260 nm.

Total collagen content was measured using the hydroxyproline assay. In brief, 100 μl of papain digested COMatrix hydrogel was added to 100 μl of 4 N NaOH separately, then hydrolyzed by autoclaving for 15 minutes. After cooling the samples to room temperature, 100 μl of 4 N HCl was added. Then, 100 μl of the resultant solution was mixed with 100 μl of chloramine-T solution and incubated for 20 minutes at room temperature. Afterward, 200 μl of p-dimethylaminobenzaldehyde (p-DAB) solution was added to the mixture, the tubes were incubated at 60° C. for 30 minutes, then quenched in room temperature water for 5 minutes. A 100 μl sample of the prepared mixture was transferred to a 96-well plate in triplicate, and the mixture absorbance was measured at 540 nm. Hydroxyproline dissolved in sodium phosphate buffer with concentrations of 200, 100, 50, 25, 12.5, 6.25, 3.125, 1.156 and 0 mg/ml was used to draw the standard curve.

The content of sGAG was measured using a 1, 9-dimethyl methylene blue (DMMB) assay. DMMB working solution was the combination of 5 mL of formate solution (0.25 g sodium formate in 24 mL of 1 M guanidine hydrochloride (GuHCl) and 0.2975 mL of >95% formic acid), 12.5 mL 200 proof anhydrous ethanol, 6.4 mg of DMMB (Sigma), and 7.5 mL ultrapure water. Papain-digested ECM (100 μl) or standard was added to 1 mL of DMMB working solution, agitated at 150 rpm for 30 minutes in room temperature, and centrifuged to precipitate as SGAG-dye complex. The supernatant was aspirated, and 1 mL decomplexation solution was added to the pellet, before being agitated, again, at 150 rpm for 30 minutes at room temperature. The samples were transferred to a 96-well plate in triplicate, and absorbance was measured at 650 nm. Serial dilutions of chondroitin sulfate (Sigma) from 200 μg/100 μl to 0 μg/100 μl were used as standards.

SDS-PAGE and Western Blot. To evaluate the quality of fabricated COMatrix hydrogels derived from De- and FT-decellularization protocols, the samples were compared to pepsin-digested bovine tendon collagen using SDS-Page electrophoresis. Each sample (7.5 μl, 6 mg/ml) was mixed with 3.75 μL LDS 4× Sample Buffer, 1.5 μL 10× Reducing Agent (dithiothreitol), and 2.25 μL DI H₂O (all were NuPAGE, Fisher Scientific) to a total volume of 15 μl. The samples were vortexed and spun down and then heated at 95° C. in a heating block for 15 minutes. Next, the samples were loaded into a 4-12% Bis-Tris gel (10 wells, 12 μl per well, NuPAGE, Fisher Scientific) and subjected to electrophoresis using SDS running buffer (NuPAGE, Fisher Scientific) and constant voltage (150 V) until the dye reached the bottom of the gel (˜1.5 hours). Deionized water (3×5 min) was used to wash the gel and stained with Bio-Safe Coomassie Brilliant Blue G-250 (50 ml per gel, BIO-RAD, USA) for 1 hour with gentle agitation. To visualize distinct bands, the stained gel was rinsed with deionized water for 30 minutes.

For western blot experiments, same concentrations of samples were separated in SDS-Page as described above, and the protein bands were transferred to polyvinylidene difluoride membranes. The membranes were gently agitated in 5% BSA in tris-buffered saline (TBS) for 1 hour at room temperature. The membranes were then gently shaken in 5% BSA in TBS containing primary antibody at 4° C. overnight. To detect the keratocan protein, the polyclonal goat anti-keratocan (Santa Cruz Biotechnology) was used with dilution of 1:1000. To detect the α-Gal epitope, a mouse monoclonal anti-Galα1-3Galβ1-4GlcNAc-R (Enzo Life Sciences) was used with dilution of 1:5. After the overnight incubation with primary antibodies, the membranes were washed with TBS containing 0.03% TWEEN®-20 for 3 times (each 10 minutes) and then incubated with the horseradish peroxidase-conjugated secondary antibodies (1:10000 dilution, Thermos Fisher) for 1 hour at room temperature followed by 4 rounds of washing with TBS/TWEEN®-20 solution. At the end, the membranes were imaged with a commercial detection system (ECL Plus Western Blotting Detection System; Amersham, Buckinghamshire, UK) using ImageQuant LAS 4000 series (GE). All data were analyzed in a semi-quantitative way using imageJ for western blot and data were normalized based on negative controls. Native cornea was not decellularized, but was fully processed, and was used as a positive control and the reference for analysis.

Ultra-Structural Characterization of Thermoresponsive COMatrix Hydrogel. The ultra-structure of thermogelated COMatrix hydrogels was observed using scanning electron microscopy (SEM). Samples (5 mg/ml) were fixed with cold glutaraldehyde overnight and then dehydrated with serial dilutions of ethanol/hexamethyldisilazane (2:1, 1:1, 1:2 and 0:1, respectively, each step 30 minutes and allowed to evaporate in the last step). After that, the samples were sputter-coated and visualized using SEM (JEOL JSM-IT500HR FESEM).

Turbidimetric Assay for Gelation Kinetics of Thermoresponsive COMatrix Hydrogels. Gelation kinetics was determined via turbidimetric spectrophotometric analysis. This technique is based on increased turbidity, and thus absorbance, experienced during hydrogel self-assembly. The sol-gel transition (thermogelation) of the thermoresponsive COMatrix hydrogels when heated to 37° C. was characterized by turbidimetric assay. In this assay, 160 μl of 25 mg/ml cool (4° C.) COMatrix hydrogel was loaded cold in a 96-well plate. The experiment was performed in triplicate and repeated three times. Then, the plate was placed in a pre-warmed (37° C.) plate reader and absorbance at 405 nm was measured every 2 minutes, for 30 minutes. Absorbance values were normalized with the following formula: NA=(A−A₀)/(Aₘₐₓ−A₀), where NA is the normalized absorbance, A is the absorbance at any given time, A0 is the initial absorbance and Amax is the maximal absorbance. The time needed to reach 50% of the maximum turbidity measurement (e.g., maximum absorbance value) was defined as t_{1/2}. The lag phase (t_{lag}) was calculated by obtaining the linear portion of the curve and extrapolating the time value at which the normalized absorbance is 0. Similarly, t_{1/2} was determined as the time at which the normalized absorbance is 0.5. The speed (S) of the gelation based on turbidimetric measurements was determined by calculating the maximum slope of the growth portion of the curve.

Light Transmission Measurements. To measure transparency of fabricated thermoresponsive COMatrix hydrogels resulting from the two different decellularization protocols, 160 μl of 4° C. De-COMatrix or FT-COMatrix hydrogels at concentrations of 25 mg/ml (each triplicate) was loaded into a 96-well plate (˜500 uμm thickness). The thermogelation was induced and completed by incubating at 37° C. for 30 minutes. Light absorbance of each well was then measured at 300 to 800 nm, in 50 nm increments, in a plate reader pre-warmed to 37° C. (BIOTEK™ Synergy™ H1 Hybrid). The absorbance of the same amount of PBS was deducted from recorded values. Then, the light transmission was calculated by using the following formula: Light Transmission (%)=antilog (2−absorbance). The same process was performed to measure the light-transmission of De-LC-COMatrix and FT-LC-COMatrix cured with green light for 4 minutes.

Rheological Kinetics and Characterization of Thermoresponsive and Light-Curable COMatrix Hydrogels. A rotational rheometer (Kinexus Ultra+, Malvern) with a parallel 25 mm plates and temperature controller were used to record the thermogelation course of COMatrix hydrogels. The initial temperature of the rheometer bed was set to 12° C., assuming gelation was negligible at this temperature. COMatrix hydrogel in 25 mg/ml concentration was loaded. The parallel gap was set to 0.9 mm and to prevent sample dryness, mineral oil was applied and trimmed around the plate for the duration of the experiment. The rheological indexes were measured with 0.159 Hz (1 rad/s) frequency and 0.5% strain. After recording for three minutes, the temperature was rapidly increased to 37° C. (in 25 seconds) to induce gelation. Gelation data were recorded for 50 minutes to characterize kinetics and measure the rheological shear moduli (elastic, G′ and viscous, G″ shear moduli). At least three different samples were characterized for each decellularization group.

For De-LC-COMatrix and FT-LC-COMatrix, a glass plate was used to settle the hydrogel on top and a light source was used from bottom. The parallel gap was set to 0.4 mm. The shear characteristics were started to record at 0.159 Hz (1 rad/s) frequency and 0.5% strain for 1 minute, and then the green light (100 μW/cm²) switched on (at minute 1), and the photogelation started while recording the shear moduli. The light was turned off at minute 5 (total 4 minutes light curing) and recording was continued for another 6 minutes.

In Vitro Cytocompatibility and Cell-Mediated Contraction of Hydrogels. Human corneal MSCs obtained from human cadaveric corneas were isolated and expanded as described elsewhere herein. MSCs were used to evaluate the biocompatibility of the fabricated thermoresponsive and LC-COMatrix hydrogels derived from De-and FT-decellularization protocols. Gelation was induced either thermally by adding 200 μl of cold thermoresponsive COMatrix hydrogel into one well of a 48-well plate (˜300 μm thickness) and incubation for 45 minutes at 37° C.; or by green light curing of LC-COMatrix hydrogels (200 μl, 300 μm) in a 48-well plate for 4 minutes. Then, MSCs (4×10³ cells/well) were seeded on top of the hydrogels, and 350 μl medium added to each well. The plates were incubated in a humidified incubator with 5% CO₂ at 37° C. To measure the cell-mediated contraction of the fabricated thermoresponsive and LC-COMatrices, the sizes of hydrogels seeded with MSCs were followed by scanning the plates using a document scanner (Epson) over different time points (Day 0, 9, 18 and 28).

The viability of MSCs was evaluated on day 28 of culture. The cells were stained with Calcein-AM (live cells), propidium iodide (PI, dead cells) and Hoechst 33342 (total cells, all from Sigma) and incubated for 1 hour at 37° C. in humidified atmosphere with 5% CO₂. Then, the seeded cells on COMatrices were imaged using ZEISS Cell Observer SD Spinning Disk Confocal Microscope (Zeiss, Germany), and the images were analyzed using ZEN Lite software (Zeiss, Germany).

Statistical Analyses. Data are shown as mean±standard deviation (SD). Statistical analyses were done by GraphPad Prism software version 8.3.0 (538) for Windows, (GraphPad Software, San Diego, CA) using Student's t-test for comparing the means between two groups and one-way analysis of variance (ANOVA) and Tukey posttest for more than two groups. P-values less than 0.05 were considered as statistically significant difference between groups.

Detergent and Freeze-Thaw-based Decellularization Methods have Similar Effects on Histology and Composition of Porcine Corneas/COMatrices. Several experiments were performed that compared the histology and composition of corneas decellularized with detergent-based or freeze-thaw methods. H&E staining showed successful removal of corneal epithelial and stromal cells in both techniques. Measuring the DNA remnant in decellularized samples confirmed significant reduction of DNA in both utilized protocols (p<0.0001). Alcian blue staining showed the presence of sGAG in both decellularized tissues, which were similar to native cornea. Both decellularization methods did not considerably change the total content of sulfated glycosaminoglycan (sGAG) in either FT-COMatrix or De-COMatrix as compared to native cornea. sGAG concentration was 0.25±0.06 mg, 0.22±0.04 mg and 0.20±0.04 mg per each mg of tissue in native cornea, FT-COMatrix and De-COMatrix, respectively (p=0.48). Lastly, the concentrations of total collagen in native cornea, FT-COMatrix and De-COMatrix after decellularization were 0.74±0.15 mg, 0.72±0.15 mg and 0.70±0.13 mg, respectively, per each mg of decellularized tissue (p=0.95). Likewise, picro-sirius red staining also showed that the collagen fibers were not influenced by decellularization protocols. In all experiments, no significant difference was observed between the two decellularization techniques.

Further analysis of the composition of decellularized samples was performed using SDS-PAGE electrophoresis. The protein bands were similar in both hydrogel samples from different decellularization methods as compared to native cornea. Also, it was clear that hydrogels from decellularized and digested porcine corneas were richer in the variety of proteins compared to digested bovine tendon collagen. Moreover, Keratocan, a protein rich in cornea with significant corneal healing effect, was conserved in both decellularization methods. Furthermore, SEM was used to visualize the ultrastructure of the hydrogels, which confirmed that the porous and fibrillary structure of hydrogels were similar following both decellularization methods.

α-Gal Epitope was Significantly Decreased after Porcine Cornea Decellularization. The α-Gal content before and after decellularization was measured using immunofluorescence staining and western blot.

Immunofluorescence staining showed that both freeze-thaw and detergent-based decellularization methods had removed the α-Gal epitope to some extent compared to native cornea. However, no α-Gal epitope was detected in the porcine corneas treated with α1-3, 6 Galactosidase at the end of decellularization process. Moreover, measuring the relative amount of α-Gal epitope by western blot showed that detergent-based and freeze-thaw decellularization methods had significantly removed α-Gal epitope compared to native cornea (p<0.001). Treatment with α1-3,6 Galactosidase further decreased the concertation of α-Gal to barely detectable levels (p<0.0001).

FT-COMatrix and De-COMatrix Hydrogels had Similar Gelation Kinetics and Light Transmittance. Gelation kinetics of both thermoresponsive hydrogels were analyzed by turbidimetric analysis. FT-COMatrix hydrogels presented a more sigmoidal profile compared to De-COMatrix hydrogels. However, the gelation kinetics of both hydrogels looked more similar after normalization. Although the thermogelation of De-COMatrix started (t_{lag}, 5.7 minutes) earlier than FT-COMatrix (t_{lag}, 10.7 minutes), there was no statistically significant difference in kinetics of gel formation between the two decellularization methods (both hydrogels had similar linear area time, slope, and gelation half-time, t_{1/2}).

All thermoresponsive and light-curable hydrogels allowed light to pass through them following thermogelation similar to human cornea (500 μm thickness for all samples). Both thermoresponsive and light-curable hydrogels derived from detergent-based decellularization technique were slightly less transparent to the naked eye, although quantitative light transmittance measurements using spectrophotometer did not show a significant difference between various hydrogels and human cornea.

COMatrix Derived from Freeze-Thaw Decellularization has Higher Rheological Shear Moduli. Rheology was utilized to assess mechanical gelation kinetics and characteristics of the thermoresponsive and LC-COMatrix hydrogels. The thermoresponsive hydrogels derived from detergent-based and freeze-thaw decellularizations were exposed to increasing temperatures from 12 to 37° C. Higher temperature induced thermogelation, as evident by increase in shear moduli, and thermogelation, which was completed in 10 minutes. The average final elastic (G′) and viscous (G″) moduli for thermoresponsive De-COMatrix were 82.4±5.5 Pa and 11.1±4.5 Pa, respectively, and 300.8±22.5 Pa and 178.7±31.3 Pa, respectively, for thermoresponsive FT-COMatrix. FT-COMatrix hydrogel had significantly higher shear moduli than De-COMatrix hydrogel (N=3, p<0.01).

The photogelation of LC-COMatrix samples was initiated by turning the light source on and was recorded by increase in the shear moduli. The achieved elastic shear moduli (G′) and viscous shear moduli (G″) after 4 minutes of light curing for FT-LC-COMatrix (25 mg/ml) were 18.3±1.7 kPa and 0.5±0.2 kPa, respectively, and 2.8±2.6 kPa and 0.3±0.2 kPa, respectively, for De-LC-COMatrix (25 mg/ml). The decellularization method had a significant effect on the biomechanical properties of hydrogels even after functionalizing to become light-curable. The light-curable hydrogels derived from FT decellularization protocol had significantly higher shear moduli compared to hydrogels derived from De decellularization protocol (N=3, p<0.0001).

Thermoresponsive and LC-COMatrix Hydrogels are Cytocompatible. Human corneal MSCs were seeded on thermoresponsive COMatrix hydrogels (FT-COMatrix or De-COMatrix) after thermogelation, or on LC-COMatrix hydrogels (FT-COMatrix or De-COMatrix) after photogelation. The tissue culture plates were scanned on days 0, 9, 18, and 28 to follow the changes in the dimensions of hydrogels. The viability of cells was evaluated at day 28 by using live-dead assay. Over 28 days in culture, the thermoresponsive hydrogels underwent significant shrinkage reflecting the ability of viable MSCs to actively contract the hydrogels (N=3), and biomechanical thermoresponsive hydrogels. Both characteristics of thermoresponsive De-and FT-COMatrices shrank significantly and there was no difference between them while comparing their surface area at day 28 of follow-up (p=0.68). The MSCs maintained viability of more than 95% after 28 days in culture on thermoresponsive hydrogels.

LC-COMatrix hydrogels were subjected to the same assays revealing notable differences in the biomechanical properties based on the decellularization method. In particular, the De-LC-COMatrix hydrogel showed considerable shrinkage on day 28 (24.5±4.1% of primary surface area) whereas the FT-LC-COMatrix hydrogel showed significant resistance to cell-mediated contraction during the same period (97.1±2.4% of primary surface area, p<0.0001). This result is consistent with the rheological data from light-curable hydrogels that revealed higher shear moduli for FT-LC-COMatrix compared to other variants of COMatrix. Live-dead assay performed on day 28 also showed high viability of MSCs on both types of LC-COMatrix hydrogels.

What is claimed is:

1. A thermoresponsive, in situ curable hydrogel composition comprising decellularized corneal extracellular matrix functionalized with an acrylate in admixture with a photo-initiator.

2. The thermoresponsive, in situ curable hydrogel composition of claim 1, wherein the acrylate is a methyl acrylate, ethyl acrylate, 2-chloroethyl vinyl ether, 2-ethylhexyl acrylate, hydroxyethyl methacrylate, butyl acrylate, butyl methacrylate or trimethylolpropane triacrylate.

3. The thermoresponsive, in situ curable hydrogel composition of claim 1, wherein said composition further comprises cells, exosomes or a therapeutic agent.

4. The thermoresponsive, in situ curable hydrogel composition of claim 1, wherein the weight ratio of decellularized corneal extracellular matrix to acrylate is in the range of 1:2 to 2:1.

5. A method for preparing the thermoresponsive, in situ curable hydrogel composition of claim 1 comprising
(a) functionalizing decellularized corneal extracellular matrix with an acrylate; and
(b) combining the functionalized, decellularized corneal extracellular matrix with a photo-initiator to provide the thermoresponsive, in situ curable hydrogel composition.

6. A kit comprising
(a) decellularized corneal extracellular matrix functionalized with an acrylate; and
(b) a photo-initiator.

7. A method for treating an ocular surface wound comprising administering the thermoresponsive, in situ curable hydrogel composition of claim 1 to the ocular surface wound and applying light to cure the hydrogel thereby treating the ocular surface wound.

8. An ocular device comprising a cured hydrogel composition comprising decellularized corneal extracellular matrix functionalized with an acrylate.

9. The ocular device of claim 8, said device having an adhesion strength of at least 10 kPa.

10. The ocular device of claim 8, said device being in the form of a lens or bandage.

11. The ocular device of claim 8, wherein said device is 3D printed.

12. A method for treating an ocular surface wound comprising administering the ocular device of claim 8 to the ocular surface wound thereby treating the ocular surface wound.
